(19) United States
(12) Patent Application Publication
(10) Pub. No.: US 2017/0290515 A1
(43) Pub. Date: Oct. 12, 2017

(54) TISSUE CLASSIFICATION METHOD USING TIME-RESOLVED FLUORESCENCE SPECTROSCOPY AND COMBINATION OF MONOPOLAR AND BIPOLAR CORTICAL AND SUBCORTICAL STIMULATOR WITH TIME-RESOLVED FLUORESCENCE SPECTROSCOPY

(71) Applicant: Cedars-Sinai Medical Center, Los Angeles, CA (US)

(72) Inventors: Pramod BUTTE, Studio City, CA (US); Chirag PATIL, Encino, CA (US); Keith L. BLACK, Los Angeles, CA (US); Fartash VASEFI, Sherman Oaks, CA (US); David Scott KITTLE, Running Springs, CA (US); Bartosz BORTNIK, Los Angeles, CA (US); Zhaojun NIE, Pasadena, CA (US)

(21) Appl. No.: 15/482,442

(22) Filed: Apr. 7, 2017

Related U.S. Application Data
(60) Provisional application No. 62/320,314, filed on Apr. 8, 2016.

(51) Int. Cl. A61B 5/00; G01N 21/64; A61B 18/24

(52) U.S. Cl. CPC A61B 5/0071; A61B 5/4064; A61B 5/7203; A61B 5/7271; A61B 18/24; G01N 21/6486; A61B 2018/00642

(57) ABSTRACT

Provided herein are methods for classifying or characterizing a biological sample in vivo or ex vivo in real-time using time-resolved spectroscopy and/or electrical stimulation. A biological sample may produce a responsive fluorescence signal when irradiated by a light excitation signal or pulse at a predetermined wavelength. The responsive fluorescence signal may be recorded. The intensity of the excitation wavelength may be recorded and used to normalize the recorded responsive fluorescence signal. The biological sample may produce a responsive electrical signal in response to electrical stimulation. Raw fluorescence decay data may be generated from the responsive fluorescence signal and pre-processed. The pre-processed raw fluorescence decay data may be de-convolved to remove an instrument response function therefrom and generate true fluorescence decay data. The biological sample may be characterized in response to the responsive fluorescence signal, the responsive electrical signal, the normalized responsive fluorescence signal, and/or the true fluorescence decay data.

### CROSS-REFERENCE

This application claims the benefit of U.S. Provisional Application No. 62/320,314, filed Apr. 8, 2016, which application is incorporated herein by reference.

### BACKGROUND OF THE INVENTION

Multiple technologies are currently available, or are under investigation, for use in distinguishing between tissue types. Such technologies may be of particular use in identifying tumor tissue during surgical resection in order to prevent unnecessary resection of healthy tissue surrounding the tumor tissue which may otherwise occur without clear identification of the tumor margins. This may be of particular importance in situations where preserving as much healthy tissue intact as possible is desired, for example in the case of brain tumors. Such currently available techniques include neuronavigation, brain functional mapping, pre-operative functional magnetic resonance imaging (MRI), intraoperative MRI, neuronavigation guided by pre-operative MRI, optical coherence tomography (OCT), tissue pathology, ultrasound, Raman spectroscopy, diffuse fluorescence spectroscopy, fluorescently-tagged extrinsic tumor markers, administered fluorescent markers such as 5-aminolevulinic acid (5-ALA), and the like. Even with so many technologies, there continues to be difficulty in identifying the exact location of a tumor and the margins of the tumor as there is often very little visual difference between tumor tissue and healthy brain tissue.

There are currently few optical technologies that aim to distinguish between tumor tissue and normal tissue during surgical resection operations. For example, OCT and Raman spectroscopy have been proposed for intraoperative use in differentiating between brain tumor tissue and normal brain tissue (see e.g., Kut, Carmen, et al., Detection of human brain cancer infiltration ex vivo and in vivo using quantitative optical coherence tomography, Science translational medicine 7.292(2015): 292ra100-292ra100; and Jermyn, Michael, et al., Intraoperative brain cancer detection with Raman spectroscopy in humans, Science translational medicine 7.274(2015):274ra19-274ra19). Raman spectroscopy techniques, while they may have high sensitivity and specificity, may be limited in their use as natural fluorescence of the brain (for example due to the presence of NADH, FAD, lipopigments, natural porphyrins, and other naturally-occurring fluorescent molecules) may obscure the Raman signal. OCT techniques have shown some utility in distinguishing between normal and tumor tissue but may be limited in sensitivity and specificity compared to Raman and other fluorescence-based techniques.

It would therefore be desirable to provide methods and systems which may allow for pre-operative, intraoperative, and/or post-operative characterization of a tissue (for example as normal or tumor) in order to determine tissue type boundaries and inform surgical procedures. Intraoperative differentiation of tumor tissue from healthy tissue may lead to reduced resection of normal brain tissue during neurosurgery for example. Additionally, particularly in the context of a brain tumor, it may be beneficial to provide the surgeon with information about the eloquent areas of the brain in order to reduce the risk of resection of such important tissues. It would therefore be desirable to provide methods and systems which may allow for pre-operative, intraoperative, and/or post-operative functional mapping of the eloquent areas of the brain in order to inform surgical procedures. It may also be desirable to combine information about the tumor margins and tissue type determined with functional mapping information of the brain to further enhance safety and better inform the surgeon during surgical resection of a brain tumor.

### SUMMARY OF THE INVENTION

The subject matter described herein generally relates to characterization of a biological sample and, in particular, to methods, systems, and devices for time-resolved fluorescence spectroscopy. The subject matter described herein relates to imaging, identifying, classifying, characterizing, and/or distinguishing between tissues including, but not limited to, cancerous and tumorous tissues.

In a first aspect, a method for classifying a biological sample of a subject is provided. The method may comprise assaying the biological sample to obtain a time-resolved fluorescence data, detecting a subtype's signature in the obtained time-resolved fluorescence data, and/or classifying the biological sample into the subtype. The biological sample may be a brain tissue. The biological sample may be isolated from the subject. The biological sample may be integral of the subject. Assaying the biological sample may comprise imaging the biological sample using a time-resolved fluorescence spectroscopy. The subtype may be a normal tissue or a tumor. The subtype's signature may comprise the subtype's spectral signature, spectro-lifetime signature, spectro-lifetime matrix (SLM), or fluorescence decay signature, or a combination thereof. Detecting the subtype's signature may comprise preprocessing, and/or denoising, and/or supersampling, and/or deconvolution optimization of the obtained time-resolved fluorescence data. Detecting the subtype's signature may comprise calculating a fluorescence impulse response function (fIRF) and/or SLM of the obtained time-resolved fluorescence data.

In another aspect, a method for identifying a tissue of a subject as being a normal tissue or a tumor is provided. The method may comprise assaying the tissue to obtain a time-resolved fluorescence data, detecting a normal tissue's signature in the obtained time-resolved fluorescence data, and identifying the tissue as being a normal tissue, and/or detecting a tumor's signature in the obtained time-resolved fluorescence data, and identifying the tissue as being a tumor.

In another aspect, a method for performing a surgery on a subject is provided. The method may comprise assaying a tissue of the subject to obtain a time-resolved fluorescence data, detecting a normal tissue's signature in the obtained time-resolved fluorescence data, identifying the tissue as being a normal tissue, and preserving the normal tissue, and/or detecting a tumor's signature in the obtained time-resolved fluorescence data, identifying the tissue as being a tumor, and removing the tumor.

In another aspect, a method for classifying a biological sample of a subject is provided. The method may comprise assaying the biological sample to obtain a time-resolved fluorescence data and/or an electrical function data, detecting a subtype's signature in the obtained time-resolved fluorescence data and/or the electrical function data, and classifying the biological sample into the subtype. Assaying the biological sample may comprise imaging the biological sample using a time-resolved fluorescence spectroscopy and/or recording the electrical activity of the biological sample.

In another aspect, a method for identifying a tissue of a subject as being a normal tissue or a tumor is provided. The method may comprise assaying the tissue to obtain a time-resolved fluorescence data and/or an electrical function data, detecting a normal tissue's signature in the obtained time-resolved fluorescence data and/or the electrical function data, and identifying the tissue as being a normal tissue, and/or detecting a tumor's signature in the obtained time-resolved fluorescence data and/or the electrical function data, and identifying the tissue as being a tumor.

In another aspect, a method for performing a surgery on a subject is provided. The method may comprise assaying a tissue of the subject to obtain a time-resolved fluorescence data and/or an electrical function data, detecting a normal tissue's signature in the obtained time-resolved fluorescence data and/or an electrical function data, identifying the tissue as being a normal tissue, and preserving the normal tissue, and/or detecting a tumor's signature in the obtained time-resolved fluorescence data and/or an electrical function data, identifying the tissue as being a tumor, and removing the tumor.

In another aspect, a system for classifying a biological sample of a subject is provided. The system may comprise a time-resolved fluorescence spectroscope and a monopolar and/or bipolar cortical and subcortical stimulator. The system may further comprise a laser configured for emitting an excitation light for the biological sample. The time-resolved fluorescence spectroscopy may be configured for analyzing fluorescence emitted from the biological sample. The monopolar and/or bipolar cortical and subcortical stimulator may be configured for stimulating the biological sample. The system may further comprise a module configured for recording the electrical function activity of the biological sample.

In another aspect, a method for classifying a biological sample of a subject is provided. The method may comprise providing any of the systems described herein, using the system to assay the biological sample to obtain a time-resolved fluorescence data and/or an electrical function data, detecting a subtype's signature in the obtained time-resolved fluorescence data and/or the electrical function data, and classifying the biological sample into the subtype.

In another aspect, a method for classifying a biological sample of a subject is provided. The method may consist of or may consist essentially of or may comprise: 1) assaying the biological sample to obtain a time-resolved fluorescence data; 2) detecting a subtype's signature in the obtained time-resolved fluorescence data; and/or 3) classifying the biological sample into the subtype. In various embodiments, assaying the biological sample may comprise imaging the biological sample using a time-resolved fluorescence spectroscopy as described herein.

In another aspect, a method for classifying a biological sample of a subject is provided. The method may consist of or may consist essentially of or may comprise: 1) assaying the biological sample to obtain a time-resolved fluorescence data and/or an electrical function data; 2) detecting a subtype's signature in the obtained time-resolved fluorescence data and/or the electrical function data; and 3) classifying the biological sample into the subtype. In some embodiments, the biological sample may be assayed to obtain a time-resolved fluorescence data. In some embodiments, the biological sample may be assayed to obtain an electrical function data. In some embodiments, the biological sample may be assayed to obtain a time-resolved fluorescence data and an electrical function data. In various embodiments, assaying the biological sample may comprise imaging the biological sample using a time-resolved fluorescence spectroscopy and/or recording the electrical activity of the biological sample. In some embodiments, assaying the biological sample may comprise imaging the biological sample using a time-resolved fluorescence spectroscopy. In some embodiments, assaying the biological sample may comprise recording the electrical activity of the biological sample. In some embodiments, assaying the biological sample may comprise imaging the biological sample using a time-resolved fluorescence spectroscopy and recording the electrical activity of the biological sample.

In another aspect, a method for identifying a tissue of a subject as being a normal tissue or a tumor is provided. The method may consist of or may consist essentially of or may comprise: 1) assaying the tissue to obtain a time-resolved fluorescence data; 2) detecting a normal tissue's signature in the obtained time-resolved fluorescence data, and 3) identifying the tissue as being a normal tissue. Alternatively or in combination, the method may consist of or may consist essentially of or may comprise: 1) assaying the tissue to obtain a time-resolved fluorescence data; 2) detecting a tumor's signature in the obtained time-resolved fluorescence data, and 3) identifying the tissue as being a tumor.

In another aspect, a method for performing a surgery on a subject is provided. The method may consist of or may consist essentially of or may comprise: 1) assaying a tissue of the subject to obtain a time-resolved fluorescence data; 2) detecting a normal tissue's signature in the obtained time-resolved fluorescence data; 3) identifying the tissue as being a normal tissue; and 4) preserving the normal tissue. Alternatively or in combination, the method may consist of or may consist essentially of or may comprise: 1) assaying a tissue of the subject to obtain a time-resolved fluorescence data; 2) detecting a tumor's signature in the obtained time-resolved fluorescence data; 3) identifying the tissue as being a tumor; 4) and removing the tumor.

In another aspect, a method for identifying a tissue of a subject as being a normal tissue or a tumor is provided. The method may consist of or may consist essentially of or may comprise: 1) assaying the tissue to obtain a time-resolved fluorescence data and/or an electrical function data; 2) detecting a normal tissue's signature in the obtained time-resolved fluorescence data and/or the electrical function data; and 3) identifying the tissue as being a normal tissue. Alternatively or in combination, the method may consist of or may consist essentially of or may comprise: 1) assaying the tissue to obtain a time-resolved fluorescence data and/or an electrical function data; 2) detecting a tumor's signature in the obtained time-resolved fluorescence data and/or the electrical function data, and 3) identifying the tissue as being a tumor.

In another aspect, a method for performing a surgery on a subject is provided. The method may consist of or may consist essentially of or may comprise: 1) assaying a tissue of the subject to obtain a time-resolved fluorescence data and/or an electrical function data; 2) detecting a normal tissue's signature in the obtained time-resolved fluorescence data and/or an electrical function data; 3) identifying the tissue as being a normal tissue; and 4) preserving the normal tissue. Alternatively or in combination, the method may consist of or may consist essentially of or may comprise: 1) assaying a tissue of the subject to obtain a time-resolved fluorescence data and/or an electrical function data; 2) detecting a tumor's signature in the obtained time-resolved fluorescence data and/or an electrical function data; 3) identifying the tissue as being a tumor; and 4) removing the tumor.

The methods and systems described herein can be used to image a sample from various subjects including, but not limited to, humans and nonhuman primates such as chimpanzees and other ape and monkey species; farm animals such as cattle, sheep, pigs, goats, and horses; domestic mammals such as dogs and cats; laboratory animals including rodents such as mice, rats, and guinea pigs, and the like. In various embodiments, the subject may have cancer and may need surgery to remove cancerous tissue, and the sample refers to the body part containing cancerous tissue. In various embodiments, the sample may be a tumor, cell, tissue, organ, or body part. In some embodiments, the sample may be isolated from a subject. In other embodiments, the sample may be integral of a subject. In accordance with the invention, the sample may comprise an infrared or near-infrared fluorophore.

In various embodiments, the sample may be a brain tissue. In various embodiments, the biological sample may be isolated from the subject. In various embodiments, the biological sample may be integral of the subject.

In various embodiments, the subtype is a normal tissue. In various embodiments, the subtype is a tumor. In some embodiments, the tumor is a nervous system tumor including, but not limited to, brain tumor, nerve sheath tumor, and optic nerve glioma. Examples of brain tumor include, but are not limited to, benign brain tumor, malignant brain tumor, primary brain tumor, secondary brain tumor, metastatic brain tumor, glioma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor.

In various embodiments, the subtype's signature comprises the subtype's spectral signature, spectro-lifetime signature, spectro-lifetime matrix, or fluorescence decay signature, or a combination thereof.

In various embodiments, detecting the subtype's signature comprises preprocessing, and/or denoising, and/or supersampling, and/or deconvolution optimization of the obtained time-resolved fluorescence data. In various embodiments, detecting the subtype's signature comprises calculating fIRF and/or SLM of the obtained time-resolved fluorescence data.

In various embodiments, the present invention provides a system for classifying a biological sample of a subject. The system may consist of or may consist essentially of or may comprise: a time-resolved fluorescence spectroscopy; and a monopolar and/or bipolar cortical and subcortical stimulator.

In various embodiments, the time-resolved fluorescence spectroscopy is configured for analyzing fluorescence emitted from the biological sample. In various embodiments, the monopolar and/or bipolar cortical and subcortical stimulator is configured for stimulating the biological sample.

In various embodiments, the system further comprises a laser configured for emitting an excitation light for the biological sample. In various embodiments, the system further comprises a module configured for recording the electrical function activity of the biological sample.

In various embodiments, the present invention provides a method for classifying a biological sample of a subject. The method may consist of or may consist essentially of or may comprise: providing a system as described herein; using the system to assay the biological sample to obtain a time-resolved fluorescence data and/or an electrical function data; detecting a subtype's signature in the obtained time-resolved fluorescence data and/or the electrical function data; and classifying the biological sample into the subtype. In some embodiments, a time-resolved fluorescence spectroscopy is used for obtaining the time-resolved fluorescence data. In some embodiments, a laser is used for obtaining the time-resolved fluorescence data. In some embodiments, a monopolar and/or bipolar cortical and subcortical stimulator is used for obtaining the electrical function data. In some embodiments, a module configured for recording the electrical function activity of the biological sample is used for obtaining the electrical function data.

In another aspect, a method for classifying or characterizing a biological sample is provided. The method may comprise characterizing the biological sample in response to a responsive fluorescence signal and/or a responsive electrical signal. The method may comprise characterizing the biological sample in response to a responsive fluorescence signal. The method may comprise characterizing the biological sample in response to a responsive electrical signal. The method may comprise characterizing the biological sample in response to a responsive fluorescence signal and a responsive electrical signal. The responsive fluorescence signal may optionally be produced by the biological sample in response to the biological sample being irradiated with a light pulse. The responsive electrical signal may optionally be produced by the biological sample in response to electrical stimulation.

In some embodiments, the biological sample may comprise cortical or subcortical tissue.

In some embodiments, the light pulse may comprise an excitation signal at a predetermined wavelength.

In some embodiments, the responsive fluorescence signal may comprise one or more of a spectral signature, spectro-lifetime signature, spectro-lifetime matrix, or fluorescence decay signature. The biological sample may be characterized in response to the one or more of the spectral signature, spectro-lifetime signature, spectro-lifetime matrix, or fluorescence decay signature.

In some embodiments, characterizing the biological sample in response to the responsive fluorescence signal and the responsive electrical signal may comprise splitting the responsive fluorescence signal into a plurality of spectral bands and characterizing the biological sample in response to the spectral bands.

In some embodiments, characterizing the biological sample in response to the responsive fluorescence signal and the responsive electrical signal may comprise determining a concentration of a biomolecule in response to the responsive fluorescence signal. The biomolecule may comprise any one or more of PLP-GAD (pyridoxal-5′-phosphate (PLP) glutamic acid decarboxylase (GAD)), bound NADH, free NADH, flavin mononucleotide (FMN) riboflavin, flavin adenine dinucleotide (FAD) riboflavin, lipopigments, endogenous porphyrins, or a combination thereof.

In some embodiments, the biological sample may be characterized as normal, benign, malignant, scar tissue, necrotic, hypoxic, viable, non-viable, or inflamed. The biological sample may be characterized as normal cortex, white matter, or glioblastoma for example.

In some embodiments, the biological sample may comprise brain tissue. The biological sample may be characterized as normal cortex, white matter, or glioblastoma, for example.

In some embodiments, the biological sample may comprise a target tissue. The target tissue may be ablated. The target tissue may be removed or ablated in response to the characterizing of the biological sample. The target tissue may be ablated by applying one or more of radiofrequency (RF) energy, thermal energy, cryo energy, ultrasound energy, X-ray energy, laser energy, or optical energy to the target tissue. The target tissue may be ablated with a probe, the probe being configured to irradiate the biological sample with the light pulse and collect the responsive fluorescence signal. The probe may be configured to be handheld. The probe may comprise a handheld probe. The probe may be robotically-controlled, for example with a commercially-available robotic surgery system.

In some embodiments, the biological sample may be irradiated with the light pulse and electrically stimulated with a probe.

In some embodiments, the biological sample may be electrically stimulated with one or more of a bi-polar or mono-polar cortical and subcortical stimulator.

In another aspect, a method for classifying or characterizing a biological sample is presented. The method may comprise pre-processing raw fluorescence decay data. The method may comprise de-convolving the pre-processed raw fluorescence decay data to remove an instrument response function therefrom. De-convolving the pre-processed raw fluorescence decay data may generate true fluorescence decay data. The raw fluorescence decay data may be generated from a responsive fluorescence signal collected from a biological sample exposed to a light excitation signal at a predetermined wavelength. The biological sample may be characterized in response to the true fluorescence decay data.

In some embodiments, pre-processing the raw fluorescence decay data may comprise removing high frequency noise.

Alternatively or in combination, pre-processing the raw fluorescence decay data may comprise averaging multiple repetitive measurements in the raw fluorescence decay data.

Alternatively or in combination, pre-processing the raw fluorescence decay data may comprise removing one or more outliers from a group of measurements in the raw fluorescence decay data, the group of measurements sharing a same temporal point. The method may optionally further comprise repeating the removing of one or more outliers for a plurality of measurement groups at different temporal points.

In some embodiments, de-convolving the pre-processed raw fluorescence data may comprise applying a Laguerre expansion to the pre-processed raw fluorescence data. Optionally, de-convolving the pre-processed raw fluorescence data may comprise optimizing one or more of a Laguerre parameter or a temporal shift of the Laguerre expansion. Optimizing the one or more of the Laguerre parameter or the temporal shift may comprise implementing an iterative search method.

Alternatively or in combination, de-convolving the pre-processed raw fluorescence data may comprise dividing and windowing one or more of the raw fluorescence decay data or the instrument response function in the Fourier domain.

In some embodiments, the biological sample may be characterized by generating a fluorescence decay function from the true fluorescence decay data and transforming the fluorescence decay function into a spectro-lifetime matrix. The biological sample may be characterized by comparing the spectro-lifetime matrix for the biological sample to a reference spectro-lifetime matrix for a tissue characterization.

In some embodiments, the biological sample may be characterized as normal, benign, malignant, scar tissue, necrotic, hypoxic, viable, non-viable, or inflamed.

In some embodiments, characterizing the biological sample may comprise determining a concentration of a biomolecule in the biological sample.

In some embodiments, the biological sample may be treated in response to the characterizing of the biological sample.

In some embodiments, the biological sample may comprise brain tissue.

In another aspect, a method for classifying or characterizing a biological sample is provided. The method may comprise recording an intensity of an excitation light pulse. A biological sample may be irradiated with the excitation light pulse at a predetermined wavelength to cause the biological sample to produce a responsive fluorescence signal. The method may further comprise normalizing the responsive fluorescence signal in response to the recorded intensity of the excitation light pulse. The biological sample may be characterized in response to the normalized responsive fluorescence signal.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of a time-resolved fluorescence spectroscopy (TRFS) system, in accordance with embodiments;

FIG. 2 shows a chart of the fluorescence emission spectra of various exemplary molecules after splitting by a demultiplexer, in accordance with embodiments;

FIG. 3 shows a schematic of a variable voltage-gated attenuator feedback mechanism, in accordance with embodiments;

FIG. 4A shows a chart of laser intensity variation over time, in accordance with embodiments;

FIG. 4B shows a schematic of a photodiode-based fluorescence signal correction mechanism, in accordance with embodiments;

FIG. 5A shows a chart of fluorescence decay data prior to applying de-noising, in accordance with embodiments;

FIG. 5B shows a chart of the fluorescence decay data of FIG. 5A after applying de-noising, in accordance with embodiments;

FIG. 6 shows a chart of lifetime standard variation at different repetition rates, in accordance with embodiments;

FIG. 7A shows a chart of fluorescence decay data prior to applying de-noising, in accordance with embodiments;

FIG. 7B shows a chart of the fluorescence decay data of FIG. 7A after applying de-noising, in accordance with embodiments;

FIG. 8 shows a chart of deconvolution optimization of alpha and temporal shift values to obtain minimum fIRF (fluorescence impulse response function) estimation error, in accordance with embodiments;

FIG. 9 shows a chart of a walking search algorithm method, in accordance with embodiments;

FIG. 10A shows a chart of averaged spectro-lifetime matrix (SLM) measured at six different wavelength bands and seven decay levels for glioma tissue, in accordance with embodiments;

FIG. 10B shows a chart of averaged SLM measured at six different wavelength bands and seven decay levels for normal cortex tissue, in accordance with embodiments;

FIG. 10C shows a chart of averaged SLM measured at six different wavelength bands and seven decay levels for white matter tissue, in accordance with embodiments;

FIG. 11A shows a chart of fluorescence decay profiles of normal cortex, white matter, and glioblastoma (GBM) tissues using six channel time-resolved fluorescence spectroscopy (TRFS), in accordance with embodiments;

FIG. 11B shows the spectral signature of the “slow” lifetime for the data shown in FIG. 11A, in accordance with embodiments;

FIG. 11C shows the spectral signature of the “average” lifetime for the data shown in FIG. 11A, in accordance with embodiments;

FIG. 11D shows the spectral signature of the “fast” lifetime for the data shown in FIG. 11A, in accordance with embodiments;

FIG. 12A shows a chart of fluorescence decay profiles of normal cortex, white matter, and glioblastoma (GBM) tissues using six channel time-resolved fluorescence spectroscopy (TRFS), in accordance with embodiments;

FIG. 12B shows the first derivative of the spectral signature of the “slow” lifetime for the data shown in FIG. 12A, in accordance with embodiments;

FIG. 12C shows the first derivative of the spectral signature of the “average” lifetime for the data shown in FIG. 12A, in accordance with embodiments;

FIG. 12D shows the first derivative of the spectral signature of the “fast” lifetime for the data shown in FIG. 12A, in accordance with embodiments;

FIG. 13 shows a flowchart of a method of tissue classification using TRFS data, in accordance with embodiments;

FIG. 14 shows a chart of lifetime variation at different concentrations of Rhodamine B (RD) and Rose Bengal (RB) in solution, in accordance with embodiments;

FIGS. 15A and 15B show fitting of the fluorescence impulse response function (fIRF) of the data collected in FIG. 14 to a bi-exponential function where the first exponential coefficients (FIG. 15A) and the second exponential coefficients (FIG. 15B) at multiple measurements correlate with individual concentrations of each component in the mixture, in accordance with embodiments;

FIG. 16 shows a chart of linear discriminant analysis (LDA) classification for normal cortex, normal white matter, and glioblastoma, in accordance with embodiments;

FIG. 17A shows a chart of LDA classification for normal cortex, normal white matter, and glioblastoma, in accordance with embodiments;

FIG. 17B shows a chart of “true or not true” LDA classification for white matter versus normal cortex used to generate the chart of FIG. 17A, in accordance with embodiments;

FIG. 17C shows a chart of “true or not true” LDA classification for normal cortex versus glioblastoma used to generate the chart of FIG. 17A, in accordance with embodiments;

FIG. 17D shows a chart of “true or not true” LDA classification for white matter versus glioblastoma used to generate the chart of FIG. 17A, in accordance with embodiments;

FIG. 18 shows a schematic of a TRFS system, in accordance with embodiments;

FIG. 19 shows a flowchart of an exemplary method of tissue classification, in accordance with embodiments; and

FIG. 20 shows a flowchart of an exemplary method of tissue classification, in accordance with embodiments.

### DETAILED DESCRIPTION OF THE INVENTION

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

All references cited herein are incorporated by reference in their entirety as though fully set forth. Unless defined otherwise, technical and scientific terms used herein have the same meaning as commonly understood by one of ordinary skill in the art to which the claimed invention belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, N.Y. 2006); Smith, March's Advanced Organic Chemistry Reactions, Mechanisms and Structure 7th ed., J. Wiley & Sons (New York, N.Y. 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one of ordinary skill in the art with a general guide to many of the terms used in the present disclosure. For references on how to prepare antibodies, see Greenfield, Antibodies A Laboratory Manual 2nd ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One of ordinary skill the art will recognize many methods and materials similar or equivalent to those described herein which could be used in the practice of the claimed invention. Other features and advantages of the claimed invention will become apparent from the following detailed description, taken in conjunction with the accompanying drawings, which illustrate, by way of example, various features of embodiments of the claimed invention. Indeed, the claimed invention is in no way meant to be limited to the methods and materials described herein. For convenience, certain terms employed herein, in the specification, examples, and appended claims, are collected here.

Unless stated otherwise, or implicit from context, the following terms and phrases include the meanings provided herein. Unless explicitly stated otherwise, or apparent from context, the terms and phrases used herein do not exclude the meaning that the term or phrase has acquired in the art to which it pertains. Unless otherwise defined, all technical and scientific terms used herein have the same meaning as commonly understood by one of ordinary skill in the art to which the claimed invention belongs. It should be understood that this invention is not limited to the particular methodology, protocols, and reagents, etc., described herein and, as such, can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those of ordinary skill in the art that terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the disclosure otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Conditions” and “disease conditions,” as used herein may include, but are in no way limited to, any form of malignant neoplastic cell proliferative disorders or diseases (e.g., tumor and cancer). In accordance with the present disclosure, “conditions” and “disease conditions” as used herein include, but are not limited to, any and all conditions involving a tissue difference, i.e., normal vs. abnormal, due to any and all reasons including but not limited to tumor, injury, trauma, ischemia, infection, inflammation, and/or auto-inflammation. Still in accordance with the present disclosure, “conditions” and “disease conditions,” as used herein include, but are not limited to, any situation where a tissue of interest (e.g., a cancerous, injured, ischemic, infected, and/or inflammed tissue) is different from the surrounding tissue (e.g., healthy tissues) due to physiological or pathological causes. Examples of “conditions” and “disease conditions” include, but are not limited to, tumors, cancers, traumatic brain injury, spinal cord injury, stroke, cerebral hemorrhage, brain ischemia, ischemic heart diseases, ischemic reperfusion injury, cardiovascular diseases, heart valve stenosis, infectious diseases, microbial infections, viral infection, bacterial infection, fungal infection, and autoimmune diseases.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant cancers, as well as dormant tumors, metastases, or micrometastases. Cancers which migrate from their original location and seed vital organs can eventually lead to the death of the subject through the functional deterioration of the affected organs. As used herein, the term “invasive” refers to the ability of the cancer to infiltrate and destroy surrounding tissue. Melanoma, for example, is an invasive form of skin cancer. As used herein, the term “carcinoma” refers to a cancer arising from epithelial cells. Examples of cancer include, but are not limited to, nervous system tumor, brain tumor, nerve sheath tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer (including but not limited to androgen-dependent prostate cancer and androgen-independent prostate cancer). Examples of brain tumors include, but are not limited to, benign brain tumor, malignant brain tumor, primary brain tumor, secondary brain tumor, metastatic brain tumor, glioma, glioblastoma (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor. Nervous system tumor or nervous system neoplasm refers to any tumor affecting the nervous system. A nervous system tumor can be a tumor in the central nervous system (CNS), in the peripheral nervous system (PNS), or in both CNS and PNS. Examples of nervous system tumor include but are not limited to brain tumor, nerve sheath tumor, and optic nerve glioma.

The term “sample” or “biological sample” as used herein denotes a portion of a biological organism. The sample can be a cell, tissue, organ, or body part. A sample can still be integral of the biological organism (i.e. in vivo or in situ). For example, when a surgeon is trying to remove a breast tumor from a patient, the sample can refer to breast tissue labeled with infrared dye and imaged with the imaging system described herein. In this situation, the sample is still part of the patient's body. A sample can be taken or isolated from the biological organism (i.e. ex vivo), e.g., a tumor sample removed from a subject. Exemplary biological samples include, but are not limited to, a biofluid sample, serum, plasma, urine, saliva, a tumor sample, a tumor biopsy, and/or tissue sample, etc. The term “sample” also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

As used herein, a “subject” means a human or animal. Usually the animal is a vertebrate such as a primate, rodent, domestic animal, or game animal. Primates include chimpanzees, cynomologous monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, feline species (e.g., domestic cat), and canine species (e.g., dog, fox, wolf). The terms “patient,” “individual,” and “subject” are used interchangeably herein. The subject may be mammal. The mammal can be a human, non-human primate, mouse, rat, dog, cat, horse, or cow, but are not limited to these examples. In addition, the methods described herein can be used to treat domesticated animals and/or pets.

“Mammal,” as used herein, refers to any member of the class Mammalia, including, without limitation, humans and nonhuman primates such as chimpanzees and other apes and monkey species; farm animals such as cattle, sheep, pigs, goats, and horses; domestic mammals such as dogs and cats; laboratory animals including rodents such as mice, rats, and guinea pigs; and the like. The term does not denote a particular age or sex. Thus, adult and newborn subjects, as well as fetuses, whether male or female, are intended to be included within the scope of this term.

A subject can be one who has been previously diagnosed with, identified as suffering from, and/or found to have a condition in need of treatment (e.g., tumor) or one or more complications related to the condition. The subject may optionally have already undergone treatment for the condition or the one or more complications related to the condition. Alternatively, a subject can be one who has not been previously diagnosed as having a condition or one or more complications related to the condition. For example, a subject can be one who exhibits one or more risk factors for a condition or one or more complications related to the condition. The subject may not exhibit risk factors. A “subject in need” of treatment for a particular condition can be a subject suspected of having that condition, diagnosed as having that condition, already treated or being treated for that condition, not treated for that condition, or at risk of developing that condition.

The methods and systems described herein can be used to image a sample from various subjects, including but not limited to, humans and nonhuman primates such as chimpanzees and other ape and monkey species; farm animals such as cattle, sheep, pigs, goats, and horses; domestic mammals such as dogs and cats; laboratory animals including rodents such as mice, rats, and guinea pigs; and the like. The subject may have cancer and may need surgery to remove cancerous tissue. In such instances, the sample may refer to the body part containing cancerous tissue. The sample may be a tumor, cell, tissue, organ, or body part. The sample may be isolated from a subject (i.e. ex vivo). In other embodiments, the sample may be integral of a subject (i.e. in vivo or in situ). The sample may comprise an infrared or near-infrared fluorophore.

The sample may be a brain tissue. The biological sample may isolated from the subject (i.e. ex vivo). The biological sample is integral of the subject (i.e. in vivo or in situ).

In various embodiments, the subtype may be a normal tissue. In various embodiments, the subtype may be a tumor. In some embodiments, the tumor may be a nervous system tumor including, but not limited to, brain tumor, nerve sheath tumor, and/or optic nerve glioma. Examples of brain tumor include, but are not limited to, benign brain tumor, malignant brain tumor, primary brain tumor, secondary brain tumor, metastatic brain tumor, glioma, glioblastoma (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified, thus fulfilling the written description of all Markush groups used in the appended claims.

Although specific reference is made to characterizing brain tissue as malignant or non-malignant, the methods, systems, and devices disclosed herein can be used with many types of biological samples including blood, plasma, urine, tissue, microorganisms, parasites, saliva, sputum, vomit, cerebrospinal fluid, or any other biological sample from which a chemical signal can be detected. The biological sample may be a solid, semi-solid, or liquid biological sample. The biological sample may comprise tissue from the prostate, lung, kidney, brain, mucosa, skin, liver, colon, bladder, muscle, breast, eye, mouth, muscle, lymph node, ureters, urethra, esophagus, trachea, stomach, gallbladder, pancreas, intestines, heart, spleen, thymus, thyroid, ovaries, uterus, lungs, appendix, blood vessel, bone, rectum, testicle, or cervix, to name a few. The biological sample may be any tissue or organ that is accessible through non-surgical or surgical techniques. The biological sample may be collected from a patient and characterized ex vivo. For example, the biological sample may be a biopsy that is analyzed in the operating room during surgery or in a pathology lab to provide a preliminary diagnosis prior to immunohistochemical analysis. Alternatively, the biological sample may be characterized in vivo. For example, the embodiments disclosed herein may be used to characterize tissue in the brain, breast, or skin, for example, to distinguish between cancerous and non-cancerous tissue prior to surgical resection.

The systems, devices, and methods disclosed herein may be used to characterize a biological sample. The biological sample may for example be characterized as normal, benign, malignant, scar tissue, necrotic, hypoxic, viable, non-viable, inflamed, or the like. The systems, devices, and methods disclosed herein may be used to assess for post-injury tissue viability, determine tumor margins, monitor cellular metabolism, monitor therapeutic drug concentrations in blood plasma, or the like. The systems, devices, and methods disclosed herein may be adapted for a variety of applications and uses depending on the biological sample and molecule(s) of interest being assayed.

Although specific reference is made to characterizing a biological sample using an emitted fluorescence spectrum, it will be understood that the systems, methods, and devices disclosed herein can be used to characterize tissue with many types of optical spectra. For example, the signal emitted by the biological sample in response to excitation with a light pulse may comprise a fluorescence spectrum, a Raman spectrum, an ultraviolet-visible spectrum, an infrared spectrum, or any combination thereof.

The following examples are intended to be purely exemplary of the invention, and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One of ordinary skill in the art may develop equivalent means or reactants without the exercise of inventive capacity and without departing from the scope of the invention.

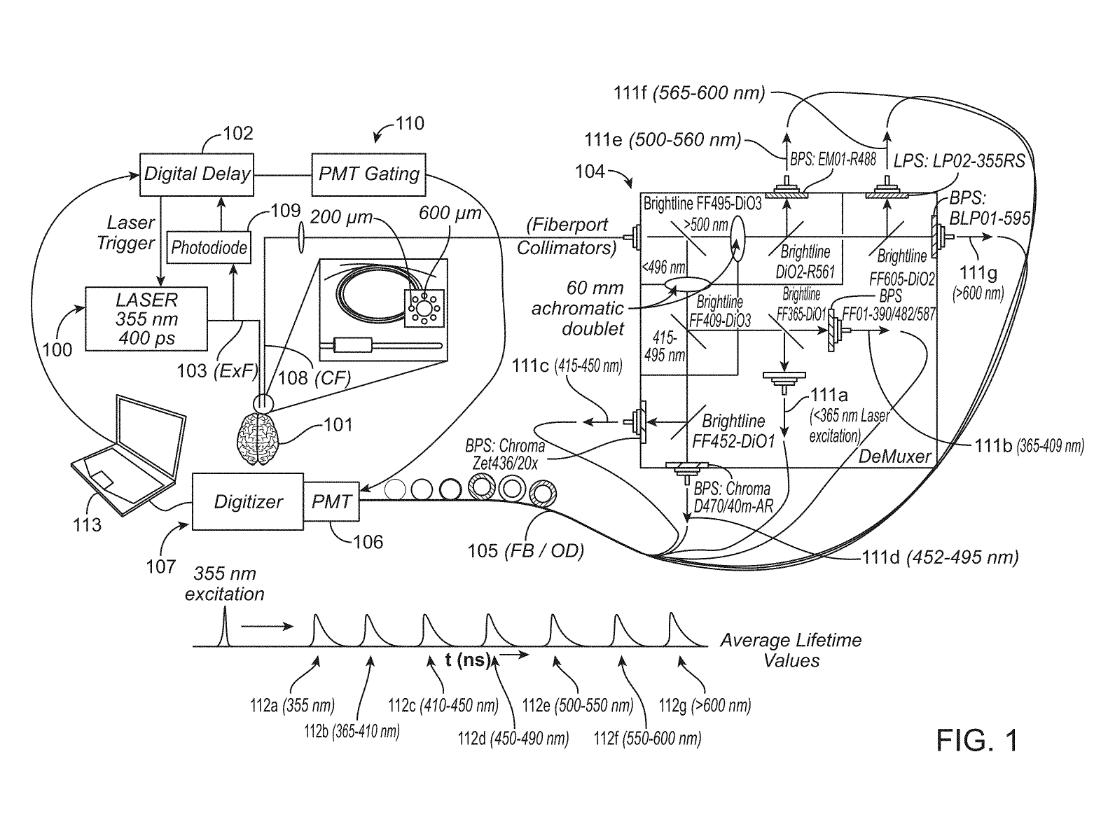

Previously, we have developed the time-resolved fluorescence spectroscopy (TRFS) system, including hardware and software technologies, which may be used to collected fluorescent information from a sample. When a laser is used to induce fluorescence in a sample, the system may be referred to as a time-resolved laser-induced fluorescence spectroscopy (TR-LIFS) system. Additional information about such systems may be found in U.S. Pat. No. 9,404,870, PCT App. No. PCT/US2014/030610; PCT App. No. PCT/US2014/029781; U.S. patent application Ser. No. 15/475,750; each of which are incorporated herein by reference in their entirety as though fully set forth. FIG. 1 shows an exemplary system which may be used to acquire a responsive fluorescence signal from a sample in order to characterize the sample as described herein.

FIG. 1 shows a schematic of a time-resolved fluorescence spectroscopy (TRFS) system. The system may be used to characterize a biological samples using real-time, or near real-time, time-resolved fluorescence spectroscopy. The system may comprise an excitation signal transmission element 103, a light source 100, at least one signal collection element 108, an optical assembly such as a demultiplexer 104, and an optical delay device or element 105. The system may further comprise one or more of a detector 106, a digitizer 107, a photodiode 109, a detector gate 110, or a trigger synchronization mechanism 102. The system may further comprise a computer or processor 113 with which the data may be processed. In some instances, at least a portion of the excitation signal transmission element 103 and the at least one signal collection element 108 may comprise a handheld or robotically-controlled probe which may operably coupled to the rest of the system components.

The light source 100 may be configured to generate a light pulse, light excitation signal, or beam of continuous light at a pre-determined excitation wavelength. For simplicity the term “light pulse” will be used herein but it will be understood by one of ordinary skill in the art that the system may alternatively or in combination utilize a continuous beam of light or light excitation signal in accordance with embodiments. The light pulse may be directed towards the biological sample 101, for example, a patient's brain, by the excitation signal transmission element 103, for example, an optical fiber. Excitation by the light pulse may cause the biological sample 101 to produce a responsive fluorescence signal which may be collected by one or more signal collection element 108. The responsive fluorescence signal may then be directed towards the demultiplexer 104 by the signal collection element 108 in order to split the responsive fluorescence signal into at least two spectral bands 111a-111g (i.e., spectral bands 111a, 111b, 111c, 111d, 111e, 111f, and 111g) at pre-determined wavelengths. The spectral bands 111a-111g may then be directed to an optical delay device 105 which applies at least one time delay to the spectral bands 111a-111g in order to temporally separate the spectral bands 111a-111g prior to being recorded. The time-delayed spectral bands 112a-112g (i.e., time-delayed spectral bands 112a, 112b, 112D, 112d, 112e, 112f, 112g corresponding to spectral bands 111a, 111b, 111c, 111d, 111e, 111f, and 111g, respectively) may then be directed towards the detector 106 and detected one at a time. For each spectral band 112a-112g, the detector 106 may record the fluorescence decay and the fluorescence intensity of a spectral band before the next spectral band reaches the detector 106. In this way, a single excitation light pulse may be used to gather both time-resolved (fluorescence decay) information as well as wavelength-resolved (fluorescence intensity) information from the responsive fluorescence signal in real-time or near real-time.

The light source 100 may comprise any number of light sources such as a pulsed laser, a continuous wave laser, a modulated laser, a tunable laser, or an LED, to name a few. The pre-determined excitation wavelength of the light source 100 may be in one or more of the ultraviolet spectrum, the visible spectrum, the near infrared spectrum, or the infrared spectrum, for example within a range of about 300 nm to about 1100 nm. The pre-determined excitation wavelength of the light source 100 may be in a range of about 330 nm to about 360 nm, about 420 nm to about 450 nm, about 660 nm to about 720 nm, or about 750 nm to about 780 nm. For example, the light source 100 may emit a light pulse at about 355 nm as shown in FIG. 1. Alternatively or in combination, the light source 100 may emit a light pulse at about 700 nm or about 710 nm. The wavelength of the light source 100 may be chosen such that the biological sample 101 produces a responsive fluorescence signal upon excitation with the light pulse. The wavelength of the light source 100 may be chosen such that the biological sample 101 produces a responsive fluorescence signal without being damaged by the light pulse. For example, ultraviolet light may be chosen to excite a wide range of fluorophores within the biological sample and can be used to excite multiple fluorophores at the same time. Prolonged exposure to ultraviolet light, however, can cause cellular damage in at least some instances. Thus, in cases where exposure to ultraviolet light is a concern, near infrared or infrared light may be a safer alternative. An infrared light source 100 may be configured to excite a similar range of fluorophores as ultraviolet light by using a two-photon (or multi-photon) technique. For example, an infrared light source 100 may be configured to emit a plurality of light pulses in very quick succession such that two photons of the light pulses simultaneously irradiate the biological sample 101. When two or more photons irradiate the biological sample 101 at the same time, their energies may be added together and the sample may produce a responsive fluorescence signal similar to that which may be produced in response to radiation with ultraviolet light but with the potential safety risk reduced.

The light source 100 may be controlled by an internal or external pulse controller device or trigger device 102 which may provide precision timing to each light pulse output by the light source 100. The timing of each light pulse may be checked using a photodiode 109 and updated using an analog to digital converter device 102, for example NI PCIe-2320. The trigger device 102 may be operably coupled to the digitizer 107 to provide feedback about the timing of the detector 106. The detector 106 may optionally be controlled by a detector gate 110 which couples the timing of the light pulse with the opening of the gate 110 and the activation of the detector 106.

The light pulse may be focused from the light source 100 into an excitation signal transmission element 103. The excitation signal transmission element 103 may guide the light pulse to expose or irradiate a pre-determined location or target tissue on the biological sample 101 with the light pulse. The excitation signal transmission element 103 may for example comprise an optical fiber, a plurality of optical fibers, a fiber bundle, a lens system, a raster scanning mechanism, a dichroic mirror device, or the like, or any combination thereof.

The light pulse may irradiate the biological sample 101 and cause the biological sample 101 to emit a responsive fluorescence signal. The responsive fluorescence signal may comprise one or more of a fluorescence spectrum, a Raman spectrum, an ultraviolet-visible spectrum, or an infrared spectrum. The responsive fluorescence signal may have a wide spectrum comprising many wavelengths. The responsive fluorescence signal may comprise a fluorescence spectrum. The responsive fluorescence signal may comprise a fluorescence spectrum and one or more additional spectra, for example a Raman spectrum, an ultraviolet-visible spectrum, or an infrared spectrum. The systems, devices, and methods described herein may be used to characterize the biological sample 101 based on the fluorescence spectrum and/or one or more additional spectra.

The responsive fluorescence signal emitted by the biological sample 101 may be collected by one or more signal collection elements 108. The signal collection element 108 may, for example, comprise an optical fiber, a plurality of optical fibers, a fiber bundle, an attenuator, a variable voltage-gated attenuator, a lens system, a raster scanning mechanism, a dichroic mirror device, or the like, or any combination thereof. The signal collection element 108 may comprise a bundle of multi-mode fibers or an objective lens, for example. The signal collection element 108 may comprise a bundle of step-index multi-mode fibers. The signal collection element 108 may comprise a bundle of graded-index multi-mode fibers. The fibers or bundle of fibers may be flexible or rigid. The signal collection element 108 may comprise a plurality of fibers which have a numerical aperture (“NA”) selected to balance between the cone angle of the light entering the signal collection element 108 and the divergence angle of the light exiting the signal collection element 108 and passing through a fiber collimator. A lower NA may increase the efficiency of the optic coupling to the delay fibers by reducing the divergence angle while a higher NA may increase the amount of signal able to be collected by increasing the cone angle.

The responsive fluorescence signal may be directed onto an optical assembly or wavelength-splitting device, for example, a demultiplexer, which splits the responsive fluorescence signal into spectral bands as described herein. For example, the responsive fluorescence signal may undergo a series of wavelength-splitting processes in the demultiplexer 104 in order to resolve the wide-band responsive fluorescence signal into a number of narrow spectral bands, each with a distinct central wavelength. The demultiplexer 104 may be configured to split the responsive fluorescence signal into any number of spectral bands depending on the number desired. For example, the demultiplexer 104 may be configured to split the responsive fluorescence signal into seven spectral bands 111a-111g in order to characterize fluorescent decay of a biological sample comprising six fluorescent molecules, with the seventh spectral band comprising the reflected excitation light.

The demultiplexer 104 may comprise one or more wavelength-splitting filter configured to split the responsive fluorescence signal at pre-determined wavelength ranges to obtain a plurality of spectral bands. The wavelength-splitting filters may comprise one or more of a neutral density filter, a bandpass filter, a longpass filter, a shortpass filter, a dichroic filter, a notch filter, a mirror, an absorptive filter, an infrared filter, an ultraviolet filter, a monochromatic filter, a dichroic mirror, a prism, or the like. The responsive fluorescence signal may undergo a series of wavelength-splitting processes in the demultiplexer 104 in order to resolve the wide-band responsive fluorescence signal into a number of narrow spectral bands, each with a distinct central wavelength. The spectral bands may be in ranges between about 370 nm to about 900 nm.

The demultiplexer 104 may, for example, be configured to split a responsive fluorescence signal into a first spectral band 111e comprising light with wavelengths in a range of about 500 nm to about 560 nm, a second spectral band 111f comprising light with wavelengths in a range of about 560 nm to about 600 nm, a third spectral band 111g comprising light with wavelengths above about 600 nm, a fourth spectral band 111c comprising wavelengths in a range of about 415 nm to about 450 nm, a fifth spectral band 111d comprising wavelengths in a range of about 450 nm to about 495 nm, a sixth spectral band 111b comprising wavelengths in a range of about 365 nm to about 410 nm, and a seventh spectral band 111a comprising wavelengths of less than about 365 nm (e.g. the excitation light). The seventh spectral band 111a which comprises the excitation light may be recorded in order to ensure accurate deconvolution of the responsive spectral bands 111b-111g.

The demultiplexer 104 may, for example, be configured to split a responsive fluorescence signal from a biological tissue sample comprising emission spectra from endogenous fluorophores. The fluorophores may, for example, comprise Flavin mononucleotide (FMN) riboflavin, Flavin adenine dinucleotide (FAD) riboflavin, lipopigments, endogenous porphyrin, free nicotinamide adenosine dinucleotide (NADH), bound NADH, or pyridoxal phosphate-glutamate decarboxylase (PLP-GAD), to name a few.

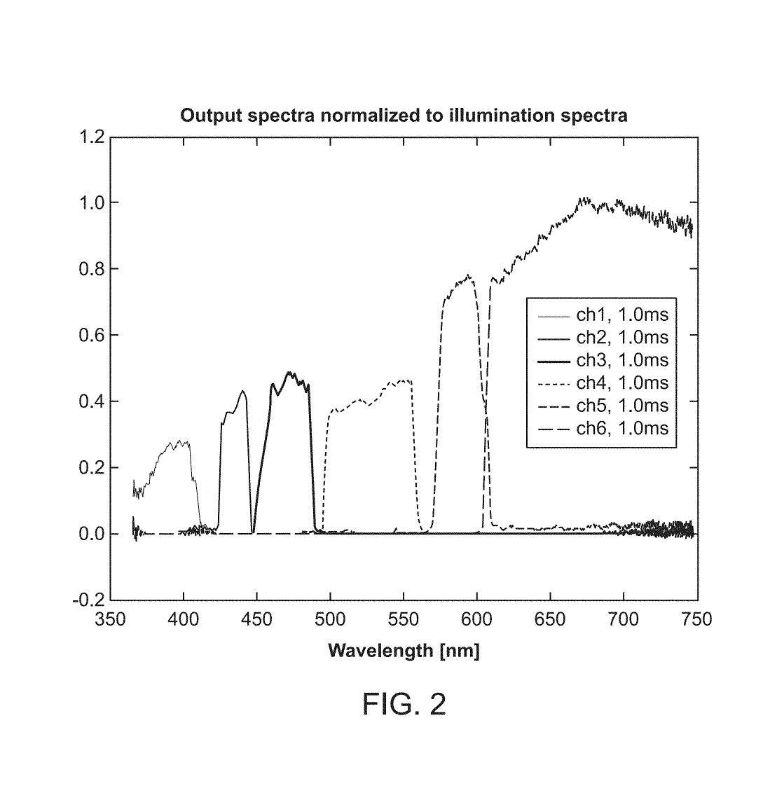

FIG. 2 shows the fluorescence emission spectra of various exemplary molecules after splitting by the demultiplexer 104. The detector 106 was used to detect the six spectral bands 111b-111g (labeled as ch1-ch6 in FIG. 2, respectively) with wavelengths above the excitation wavelength of 355 nm after a time delay was applied to each spectral band 111a-111g as described herein. The demultiplexer 104 separated the spectral bands representing PLP-GAD or purine nucleoside phosphorylase (PNP) (channel 1), bound NADH (channel 2) free NADH (channel 3), FMN/FAD/Riboflavin (channel 4), Lipopigments (channel 5), and endogenous porphyrins (channel 6). The spectral band 111a with wavelengths at or about the excitation wavelength was used to normalize the data shown.

The demultiplexer 104 may be configured to split the responsive fluorescence signal into more or fewer spectral bands as desired. In another example, the demultiplexer 104 may be configured to split the responsive fluorescence signal from a biological sample comprising free and bound NADH and PLP-GAD. The biological sample may be excited by an ultraviolet light pulse of about 355 nm as described herein. The spectral bands may be in ranges of about 400 nm or less, about 415 nm to about 450 nm, about 455 nm to about 480 nm, and about 500 nm or greater. The responsive fluorescence signal may be directed from the signal collection element onto a first wavelength splitting filter which splits the responsive fluorescence signal into a first spectral component comprising wavelengths greater than about 400 nm and a first spectral band comprising wavelengths less than about 400 nm (e.g., excitation light). The first spectral component may be split by a second wavelength splitting filter into a second spectral component comprising wavelengths in a range of about 400 nm to about 500 nm and a second spectral band comprising wavelengths greater than about 500 nm. The second spectral component may be split by a third wavelength splitting filter into a third spectral band comprising wavelengths in a range of about 400 nm to about 450 nm, for example, about 415 nm to about 450 nm, and a fourth spectral band comprising wavelengths in a range of about 450 nm to about 500 nm, for example, about 455 nm to about 480 nm.

In another example, a 440 nm light source may be used to excite a biological sample and the demultiplexer may be configured to split the responsive fluorescence signal into spectral bands for the characterization of FAD, FMN, and porphyrins.

It will be understood by one skilled in the art that the spectral bands may be in any ranges desired in order to characterize a biological sample and the wavelength splitting filters of the demultiplexer 104 may be configured to generate said spectral bands.

While an ultraviolet light pulse is described herein, it will be understood by one skilled in the art that the light source and light pulse may be any wavelength desired and the demultiplexer 104 may be configured to accommodate any wavelength of excitation light. For example, when an infrared light source is chosen, the demultiplexer 104 may be configured to split the responsive fluorescence signal into spectral bands characteristic of the biological sample and a spectral band comprising the reflected infrared light.

Referring again to FIG. 1, the wavelength-resolved spectral bands may be directed from the demultiplexer 104 to the detector 106 by the optical delay element 105. The optical delay device 105 may apply one or more time-delays to the spectral bands such that they are temporally separated and each of the time-delayed spectral bands may reach the detector 106 at different times. The optical delay device 105 may provide a delay of within a range of about 5 ns to about 700 ns. For example, the optical delay device 105 may provide one or more delay of about 7.5±3 ns, 75±10 ns, 150±10 ns, 225±10 ns, 300±10 ns, 375±10 ns, 450±10 ns, 525±10 ns, 600±10 ns, or combinations thereof. The optical delay device 105 may be configured to provide any delay or combination of delays desired. The optical delay device 105 may comprise any number of delay devices. The optical delay device 105 may comprise a plurality of optical fibers of differing lengths, one for each spectral band, such that each spectral band travels a different distance and thus a different amount of time along the optical fiber before reaching the detector 106. For example, the optical delay device 105 may comprise two optical fibers, with the second optical fiber being longer than the first optical fiber such that a first spectral band reaches the detector 106 before a second spectral band. Alternatively or in combination, physical properties of the optical fibers other than the length may be varied in order to control the time delay applied by the optical delay element 105. For example, the refractive index of the fibers may be varied. Such physical properties may also be useful in determining the length of fiber necessary to achieve a desired delay. The length of the fibers may be selected based on the delay desired. The fibers may, for example, be configured such that the lengths of fibers increase from the first to the last in increments of about 30 feet, about 35 feet, about 40 feet, about 45 feet, or about 50 feet. The increment between fibers of the optical delay device 105 may be the same or may vary between fibers. It will be apparent to one skilled in the art that any number and any lengths of fibers may be chosen in order to apply the desired temporal delay to the spectral bands. For example, the spectral bands 111a-111g may be directed towards the detector 106 by fibers with lengths of about 5 feet, 55 feet, 105 feet, 155 feet, 205 feet, 255 feet, and 305 feet, with each spectral band moving along a different optical fiber, which apply varying temporal delays to the spectral bands 111a-111g such that the time-delayed spectral bands 112a-112g reach the detector 106 at different times. Given that each spectral band may have a decay profile that last for a specific amount of time (e.g., on the order of tens of nanoseconds), the temporal delay applied to each spectral band may be configured to be sufficiently long enough to temporally separate the respective decay profiles and allow the detector to detect multiple time-delayed spectral bands after a single excitation of the biological sample 101.

The plurality of optical fibers of the optical delay device may comprise a bundle of step-index multi-mode fibers. The plurality of optical fibers of the optical delay device may comprise a bundle of graded-index multi-mode fibers. In some instances, graded-index fibers may be preferred over step-index fibers as they generally have less loss of bandwidth with increased fiber length and may thus produce a stronger or better quality signal when long fibers are used as in the optical delay devices described herein. The fibers or bundle of fibers may be flexible or rigid.

The detector 106 may be configured to receive the time-delayed spectral bands from the optical delay device 105 and record each time-delayed spectral band individually. The detector 106 may, for example, comprise a fast-response photomultiplier tube (PMT), a multi-channel plate photomultiplier tube (MCP-PMT), an avalanche photodiode (APD), a silicon PMT, or any other photodetector known in the art. The detector may be a high gain (e.g. 10⁶), low noise, fast rise time (e.g. about 80 picoseconds) photodetector, for example a Photek 210. The gain of the detector 106 may be controlled automatically. The voltage of the detector 106 may be dynamically changed based on the strength of the responsive fluorescence signal detected. The voltage of the detector 106 may be altered after analyzing the strength of the spectral bands detected and prior to recording the signal. The recorded data may be digitized for display on a computer or other digital device by a high-speed digitizer 107. The digitizer 107 may, for example, digitize the recorded data at a rate of about 6.4 G samples/second. The digitizer 107 may, for example, be a 108ADQ Tiger. The data may optionally be analyzed by a processor 113, for example, a computer processor. The processor 113 may be configured with instructions to collect the data from the digitizer 107 and perform any of the methods for analysis described herein. Alternatively or in combination, the recorded data may be displayed using an oscilloscope. An optional preamplifier may provide additional gain to the recorded data prior to display. The detector 106 may be operably coupled to a detector gate 110 which controls the detector 106 such that the detector 106 responds to signals during a narrow detection window when the detector gate 110 is open and the detector 106 is active.

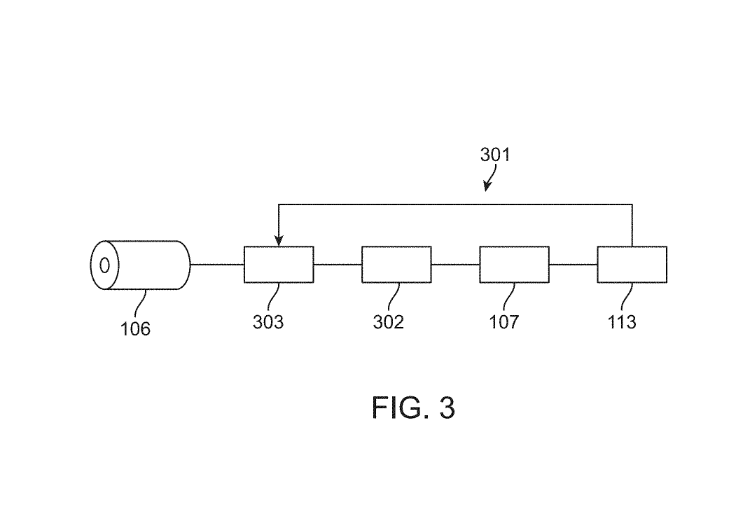

The system may optionally further comprise a variable voltage-gated attenuator 303 as shown in FIG. 3. The attenuator 303 may be operably coupled between the detector 106 and the digitizer 107. The system may further comprise a pre-amplifier 302 between the attenuator 303 and the digitizer 107. The attenuator 303 may be used to attenuate the responsive fluorescence signal before it reaches the detector 106. For example, in cases where the responsive fluorescence signal is strong enough to saturate the detector 106 and/or digitizer 107, attenuating the signal may be useful to bring the signal into a range below the saturation level of the detector 106 and/or digitizer 107 so that it may be detected and/or digitized. The attenuator 303 may attenuate the signal according to the amount of voltage applied to it. For example, if the detector 106 is saturated, a voltage may be applied to the attenuator 303 which may then attenuate the signal by a predetermined amount (which may correlate to the amount of voltage applied) in order to bring the signal below the saturation level of the detector 106. After the signal has been detected by the detector 106, the pre-amplifier 302 may amplify the responsive fluorescence signal in order to use the complete dynamic range of the digitizer 107 without affecting the signal to noise ratio (which may be a function of a gain applied to the signal by the detector 106, for example). The processor 113 may receive a signal from the digitizer 107 which may be used to modulate the activity of the attenuator 303 in a feedback-control mechanism 301. The feedback-control mechanism 301 may for example be used to adjust the voltage applied to the attenuator 303 in order to attenuate the responsive fluorescence signal in response to saturation of the detector 106 and/or digitizer 107. In some cases, saturation of the detector 106 and/or digitizer 107 may result in no responsive fluorescence signal being detected and the lack of a signal being detected at the processor 113 may trigger the feedback-control mechanism 301. In some cases, the processor 113 may detect the responsive fluorescence signal and determine if the signal is saturated, in which case the feedback-control mechanism 301 may be triggered to adjust the voltage of the voltage-gated attenuator 303.

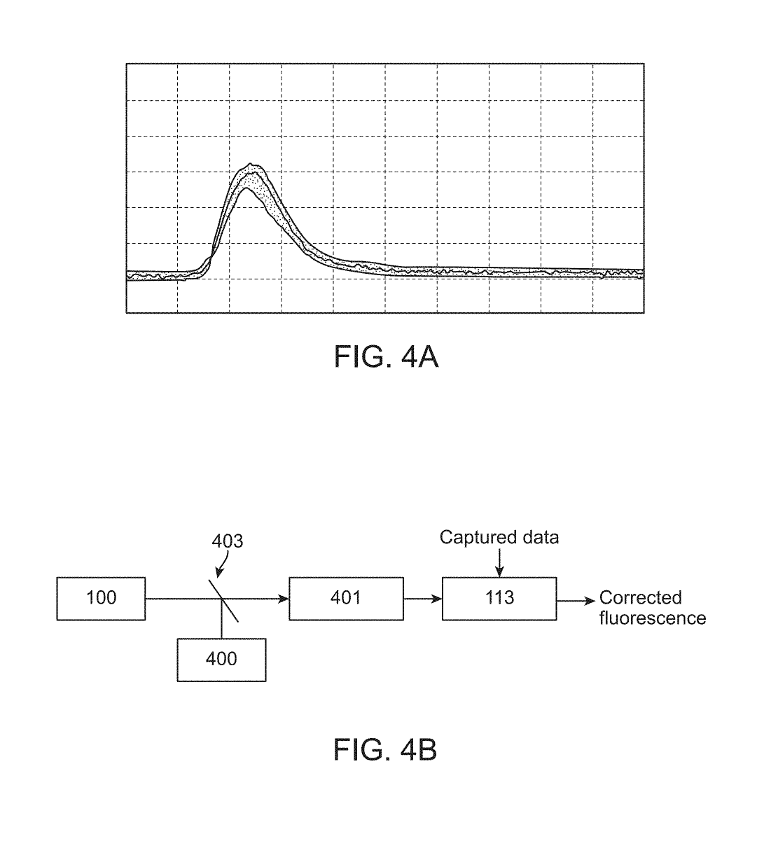

FIG. 4A shows a chart of laser intensity variation over time. The pulse intensity is shown as a mean (solid line) bounded by minimum and maximum values (grey) over time (in ns) between pulses. The pulse-to-pulse laser intensity may vary about 3% to about 5% with a typical laser system. Such variation may lead to corresponding variations in the fluorescence signal captured by the detector. When multiple responsive fluorescence signals are generated by excitation with multiple laser pulses, averaging the data collected from the responsive fluorescence signal may tend to add error to the data. To reduce this effect, the system may further comprise a photodiode-based fluorescence signal correction mechanism as shown in FIG. 4B. A photodiode 401 may be operably coupled between the light source (e.g., laser) 100 and the computer 113 in order to measure the intensity of each pulse of the laser and optionally correct the recorded responsive fluorescence signal (e.g., time-delayed spectral bands) for variations due to varying laser intensities. A beam splitter 403, or the like, may for example be used to direct a portion of the excitation light pulse towards the photodiode 401 instead of towards the TRFS probe or device 400. The intensity of each excitation light pulse may be recorded and may be used to normalize the responsive fluorescence signal of each pulse, thereby improving the accuracy of the responsive fluorescence signal. This normalized responsive fluorescence signal may be used to characterize the biological sample as described herein.

The responsive fluorescence signal from the biological sample may vary depending on the molecule of interest being excited. The responsive fluorescence signal may, for example, be very high for a highly responsive, or highly fluorescent, molecule in the biological sample or very low for a less responsive, or less fluorescent, molecule in the biological sample. A fluorophore, for example, emits a fluorescence spectrum with an intensity based on the quantum efficiency and/or absorption of the excitation light used to excite it. Depending on the conditions in which the fluorophore exists, the intensity of the fluorophore may differ. For example, a fluorophore in a tissue sample may have a different intensity than the same fluorophore in a blood sample or when isolated due to the differences in its surroundings. In order to properly record the fluorescence spectrum, the gain of the detector may be adjusted such that high fluorescence emission does not saturate the signal and low fluorescence emission does not reduce the signal to noise ratio. This may be achieved by rapidly changing the voltage of the detector 106, for example, a PMT, based on previously recorded data. For example, the biological sample may be excited with two light pulses and the recorded data may be averaged and analyzed to determine if the signal from the biological sample is too high or too low. The voltage may then be adjusted based on the determination in order to change the gain of the detector 106. Such adjustments may be done manually or automatically, for example, by the processor. Such adjustments may be done iteratively until the desired signal to noise ratio is reached. The data may be recorded once the desired signal to noise ratio is reached.

The TRFS systems and methods described herein and elsewhere may be used to generate fluorescence emission data to classify different biological tissues.

The TRFS systems and methods described herein may allow for real-time (or near real-time) data acquisition up to 1000 pulse repetitions. During data acquisition, the fluorescence emission signal may be spectrally resolved at 6 distinguished spectral bands as described herein.

In various embodiments, fluorescence emission data generated by the TRFS system described herein may be used to classify different biological tissues based on their spectro-lifetime signatures. In various embodiments, methods of data processing for the TRFS system and methods of using the TRFS system to detect different biological tissues including cancers and tumors are provided. The systems and methods described herein may improve the accuracy of biological tissue classification by reducing or removing high temporal variation of fluorescence emission measurements with limited signal-to-noise ratio.

The methods described herein may differentiate between different biological samples by analyzing light emissions from the biological sample in response to an excitation signal (e.g., a laser).

The methods described herein may include, but are not limited to, steps of: i) signal preprocessing (e.g., denoising), ii) fluorescence emission decay supersampling and/or deconvolution optimization, and iii) classification of biological tissues based on spectro-lifetime data. Various methods described herein may increase the accuracy of tissue classification by increasing fluorescence measurement repetition, removing sub-sampling limitation, and/or optimizing deconvolution processing.

In various embodiments, the tissue may be classified into a tissue subtype based on a spectral signature of the subtype. The subtype's signature comprises the subtype's spectral signature, spectro-lifetime signature, spectro-lifetime matrix, or fluorescence decay signature, or a combination thereof.

In various embodiments, detecting the subtype's signature comprises preprocessing, and/or denoising, and/or supersampling, and/or deconvolution optimization of the obtained time-resolved fluorescence data. In various embodiments, detecting the subtype's signature comprises calculating fIRF and/or SLM of the obtained time-resolved fluorescence data.

The systems and methods described herein may generally relate to methods for differentiating between biological materials (e.g. tissue types, biomolecules, etc.). Differentiation may occur by analyzing laser-induced fluorescence signal emissions from different biomolecules within the biological samples. For example, different biological samples may be differentiated by analyzing fluorescence signal emissions from the biological sample in response to a light excitation signal. The light emitted may have a fluorescence decay response at different wavelengths which is dependent on the structure of the biomolecules (such as metabolites, proteins, vitamins), or by external attachment of non-biological fluorescence agent to the biomolecule structure which may have a unique decay signature response. In many embodiments, time-resolved measurements of fluorescence decay may be emitted from the biological sample in multiple wavelengths and may, for example, be used to differentiate between at least two types of tissue. For example, the systems and methods described herein may be used for intraoperative, non-invasive, in vivo classification of a tissue sample as tumor tissue or normal tissue. The methods described herein may comprise three main stages: i) signal preprocessing, ii) deconvolution optimization, and iii) post processing classification to identify a tissue type of a biological sample.

### Signal Pre-Processing (De-Noising)

The time-delayed spectral bands may comprise raw fluorescence intensity decay data which can be measured by the systems, devices, and methods described herein. The raw fluorescence intensity decay data may be digitized by a digitizer as described herein, for example by a limited bandwidth A/D converter, which may, in some instances, lead to unwanted temporal variations between individual pulses. Such variations in the fluorescence decay data between pulses may be on the order of about 10 to about 100 picoseconds and may be due to sub-sampling and/or low signal to noise ratio (SNR). Alternatively or in combination, low tissue fluorescence intensity of the biological sample may lead to a lower SNR in some cases, which may lead to degradations in the quality of the recorded signal/decay. These variations and degradations may lower the signal quality significantly enough to affect the reproducibility and accuracy of the fluorescence lifetime measurements and may obfuscate the differences between tissue samples.

The methods described herein may be used to improve the accuracy of measurements, even when SNR is low. The raw fluorescence intensity decay data may be “pre-processed” prior to deconvolution (which may be used to remove an instrument response function (IRF) from the raw fluorescence intensity decay data to generate true fluorescence decay data) as described herein. Pre-processing may for example include removal of high-frequency noise (also referred to herein as de-noising), averaging multiple repetitive measurements in the raw fluorescence decay data, and/or removing one or more outliers from a group of measurements in the raw fluorescence decay data.

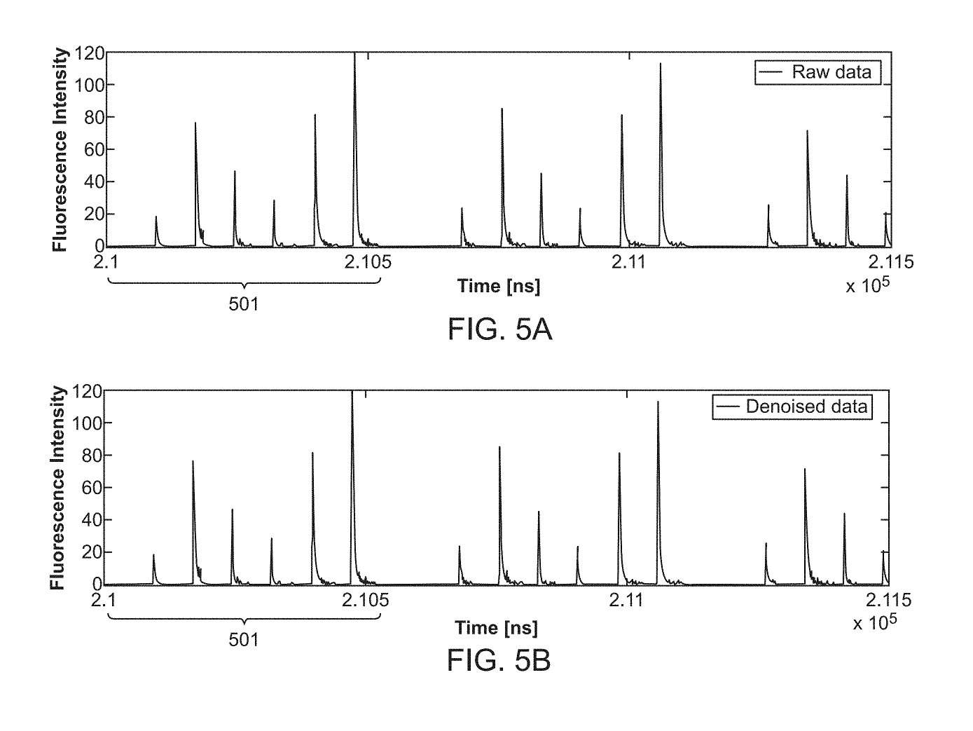

FIGS. 5A and 5B show the results of de-noising using a Savitzky-Golay filter to de-noise the raw fluorescence decay data. FIG. 5A shows a chart of fluorescence decay data prior to applying de-noising. FIG. 5B shows a chart of the fluorescence decay data of FIG. 5A after applying de-noising. The fluorescence decay data may comprise one or more sets 501 of time-delayed spectral bands generated by one or more light pulses, respectively. Each spectral band may comprise raw fluorescence decay data. The data shown here was generated using the six-channel TRFS system described herein and therefor comprises six time-delayed spectral bands, each of which comprises a raw fluorescence intensity decay signal. In some instances, multiple repetitions or pulses may be recorded over time as shown. The recorded raw fluorescence intensity decay signal may be filtered using a de-noising filter, such as a Savitzky-Golay filter, to remove high frequency noise as shown. It will be understood by one of ordinary skill in the art, however, that other filters may be used to de-noise the raw fluorescence decay data as desired.

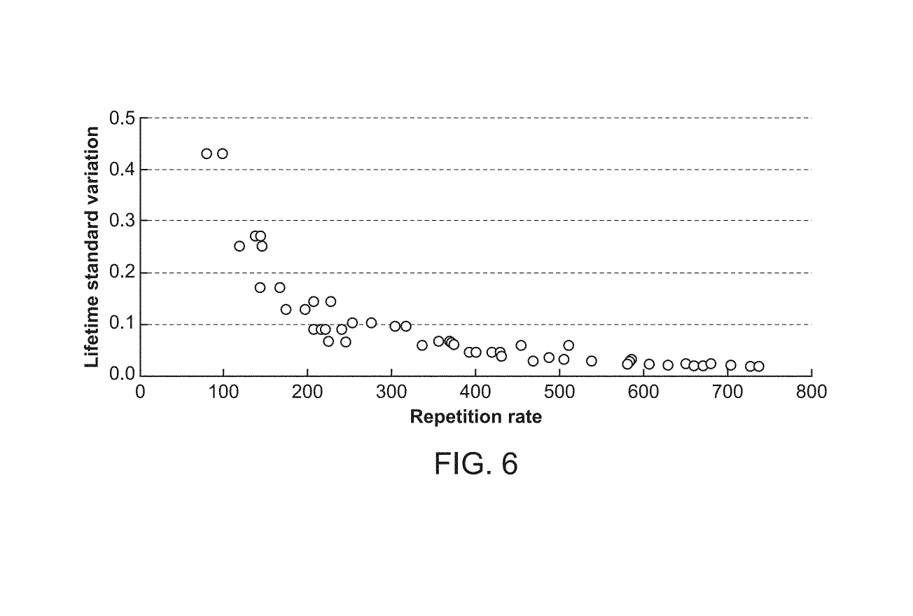

FIG. 6 shows a chart of lifetime standard variation at different repetition rates. In addition to, or as an alternative to, filtering, the raw fluorescence decay data may be averaged over multiple repetitive measurements in order to reduce signal variation and discrepancies in the signal. As the number of repetitions increases, the lifetime standard variation may be reduced as shown. For example, averaging the raw fluorescence decay data from about 1000 pulses may significantly reduce the lifetime standard variation as shown. The number of repetitions needed to reduce the variation may be dependent on a number of factors including the temporal resolution of the digitizer and the SNR.

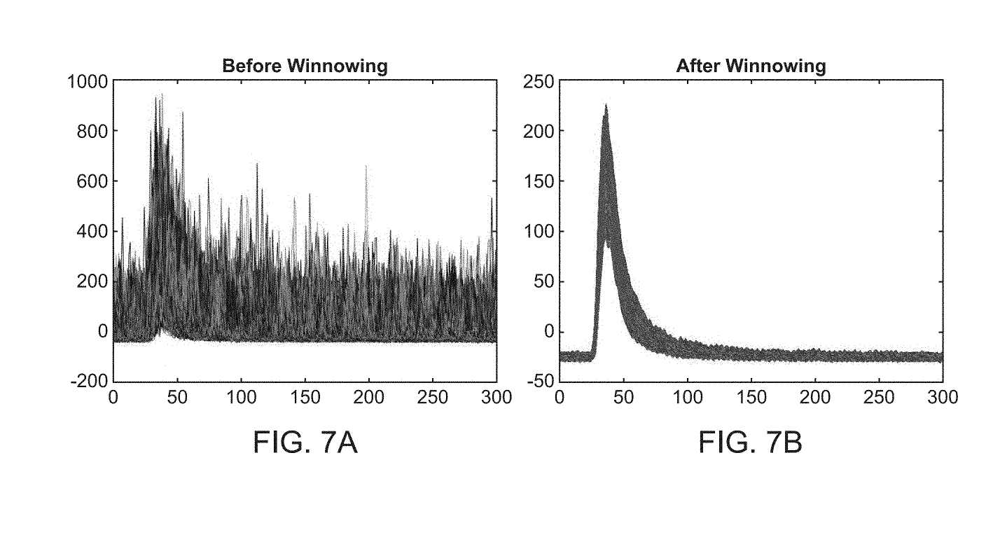

FIGS. 7A and 7B show the results of de-noising using winnowing in order to de-noise a raw fluorescence decay signal. FIG. 7A shows a chart of fluorescence decay data prior to applying de-noising. FIG. 7B shows a chart of the fluorescence decay data of FIG. 7A after applying de-noising. A single raw fluorescence decay signal for a single spectral band is shown for clarity but it will be obvious to one of ordinary skill in the art that multiple signals from multiple spectral bands and/or multiple pulse repetitions may be collected and processed as described herein. Optoelectronic systems, particularly those that utilize a photomultiplier tube (PMT) as a detector, may be subject to multiple sources of noise including shot noise and photon noise (which may be seen as spikes in the measured waveform). The impact of such noise may be diminished, and a higher SNR may be recovered, by capturing repeated measurements and averaging those measurements. While effective, such techniques may require substantial averaging of multiple collected measurements when SNR is low and may take a substantial amount of time to complete. Additionally, the bias of photodetected signals may have a tendency to increase the magnitude of the photodetected signal floor. To address this, a paradigm-shifting winnowing technique may be used to pre-process the data. Instead of performing statistical operations on collections of entire waveforms, statistical operations may instead be performed on the sample distribution composed of a particular temporal point in each of the measured waveforms. This may then be repeated for each temporal point. By treating each temporal point (found in each of the measured waveforms) as a sample distribution, statistical processes such as outlier identification can be utilized to remove sources of noise. The impact of outliers on the averaged signal may thus be reduced and fewer measurements may be used to obtain a similar SNR vis-à-vis averaging (thereby decreasing overall measurement time).

### Super-Sampling and Deconvolution Optimization

The time-delayed spectral bands may comprise fluorescence intensity decay data which can be measured by the systems, devices, and methods described herein. The measured fluorescence intensity decay data (FID(t,λ)) may be comprised of fluorescence decay components from one or more biomolecules as well as the optical and electronic transfer component functions known as Instrument Response Function (IRF(t, λ). Mathematically, the FID(t, λ) is the convolution of the fluorescence impulse response function (ƒIRF(t, λ)) with the IRF(t,λ). In order to estimate pure ƒIRF(t, λ) of a sample, the IRF(t, λ) may be deconvolved from the measured fluorescence pulse. Deconvolution may be applied to the raw fluorescence decay signal or to a pre-processed raw fluorescence decay signal. The IRF(t, λ) describes the effects of optical path and wavelength system characteristics experienced by fluorescence photons and may be measured by recording very fast fluorescence decay(s) from standard dyes. The measured fast fluorescence decay may be employed as an approximation of the true IRF(t, λ) when the decay is an order of magnitude faster than the fluorescence decay from the biological sample of interest (e.g. less than 70 ps is fast enough when brain tissue is the sample of interest). There are many mathematical models which may be used to perform deconvolution.

The “Laguerre expansion of kernels” may for example be used to determine the ƒIRF(t,λ) of the raw (or pre-processed raw) fluorescence decay data. The Laguerre method is based on the expansion of orthonormal sets of discrete time Laguerre functions. The Laguerre parameter α (0<α<1) determines the rate of exponential (asymptotic) decline of the discrete Laguerre functions. The choice of parameter α is important in achieving accurate ƒIRF(t, λ) estimations. An iterative process may be used to determine the optimal α to recover accurate fluorescence decay. Prior to estimating α and fitting the Laguerre kernels to the fluorescence decay measured, the previously-recorded IRF and the fluorescence decay may be temporally aligned. Alignment may be achieve by taking a super-sample of both IRF(t, λ) and the measure FID(t, λ). The temporal shift for deconvolution may be iteratively determined with a minimal error. The repeated measured fluorescence intensity decays (FID(t,λ)) may be averaged to correct the temporal variations due to under-sampling as described herein. The signal may then be interpolated to higher sampling rate. A common super-sampling up-conversion range can be from about two to about 100, for example about 10. The super-sampling up conversion accuracy may be dependent on signal-to-noise level and number of repetitions.

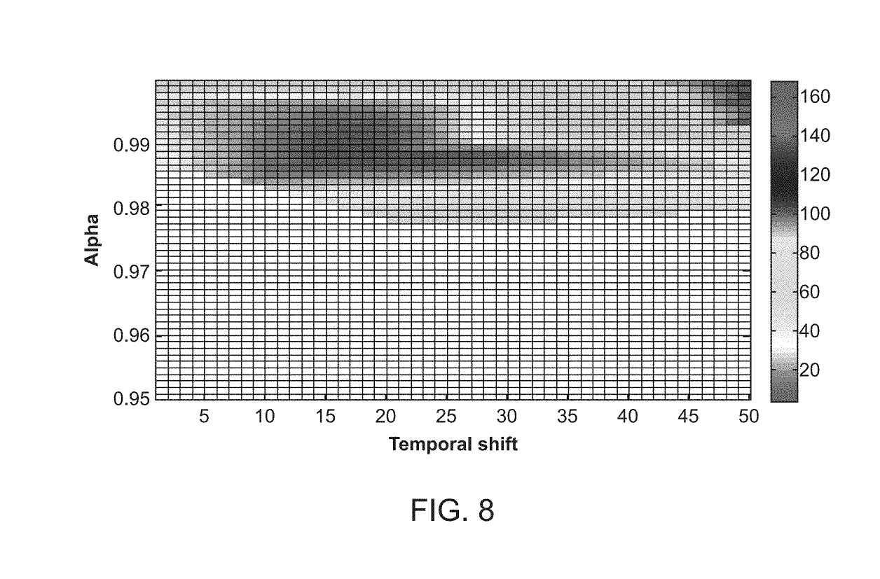

FIG. 8 shows an optimization search method for finding values for α and the temporal shift. The method may be used to determine values for α and the temporal shift for a given signal. The particular values determined for α and the temporal shift may be dependent on the digitizer (and the sampling rate used) and/or the sample's decay profile. The fluorescence decay response fIRF(t,λ) is monotonically decreasing, convex, and asymptotically ends to zero. This suggests that two conditions need to be fulfilled by the values for α and the temporal shift during deconvolution searches for the fIRF(t,λ). First, the first derivative should have a negative value. Second, the second derivative should have a positive value. The white areas in FIG. 8 show the fIRF(t,λ) which do not pass the first and second derivative conditions. In some instances, global search method and/or a random walk method were used to obtain optimized α and temporal shift values. The global search method may search through all combinations of α and the temporal shift, whereas the random walk method may search fewer combinations based on the assumption that a single minimum exists. It will be understood by one of ordinary skill in the art that other search algorithms may be used to determine values for α and the temporal shift as desired.

Using a global search algorithm method, a range of α and temporal shift values may be scanned and used to calculate deconvolution and a deconvolution error estimation for each α and temporal shift value. The α and temporal shift ranges scanned may be pre-defined, for example based on prior knowledge of optimized values. The deconvolution calculation can be done in parallel with processing in order to minimize the total processing time.

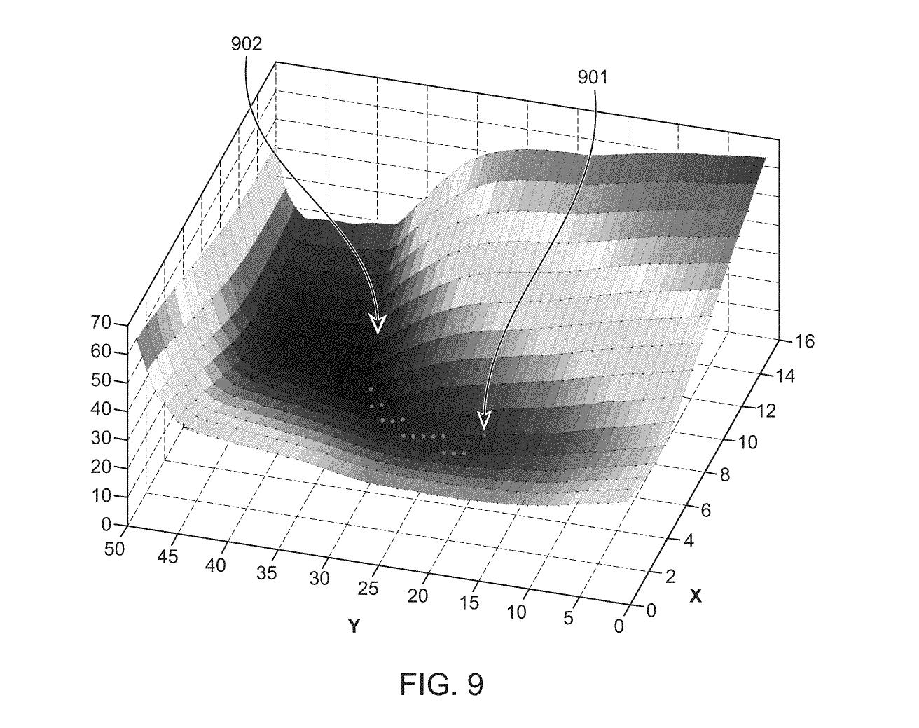

A walking search algorithm may be used to rapidly find a global minimum. Assuming a convex function (for example a function where its epigraph is a convex set such as the quadratic function or an exponential function), where by definition a single minimum exists and is traceable from any location on the function, as in FIG. 9, a global minimum 902 can be found within a few steps by searching from an initial guess 901. From this start point 901, eight surrounding points on the function may be calculated and the gradient from the initial point 901 may be maximized and then chosen as the next location on the function surface. The algorithm may continue until the current point is lower than all surrounding eight points.

FIG. 9 shows an algorithm traversing an error function (pre-calculated to show the surface) from a time-resolved fluorescence spectroscopy measurement. The x-axis and y-axis are a and temporal shift values in a matrix when calculating the deconvolution via the IRF of the system as described herein. The initial guess 901 is shown and the final answer 902 is shown at the end of the traverse. Note that the traverse can progress along diagonals as well as x-y parallel paths. Fourteen steps were required to reach the minimum 902. The actual number of calculations of the error function for each location may be fewer that nine in most cases (other than the first location 901), because each step may re-use previous calculations. The operations used in FIG. 9 are outlined in Table 1.

TABLE 1
Number (“No.”) of calculations
for each step taken towards reaching a global minimum.

| Step No. | No. of Calculations |
|---|---|
| 0 | 9 |
| 1 | 6 |
| 2 | 3 |
| 3 | 3 |
| 4 | 6 |
| 5 | 3 |
| 6 | 3 |
| 7 | 3 |
| 8 | 3 |
| 9 | 6 |
| 10 | 3 |
| 11 | 3 |
| 12 | 6 |
| 13 | 3 |
| 14 | 6 |
| Total | 66 |

In step 0, nine locations of the error function are calculated. The next step (step 1) was diagonal to step 0 and therefore required the calculation of only six locations, as three of the locations overlapped with those calculated in step 0. For most of the other steps, only three new locations must be calculated since the other six often overlap with the previous step. The total number error function calculations for this search was 66, compared to 800 (50×16 matrices) which may have occurred by calculating the entire effort function. Such a method may thus yield a 12× speedup in the algorithm with little to no loss in accuracy. Note that this initial guess 901 was far from the final minimum 902. In many cases, the initial guess may be quite close and such techniques may therefore yield greater speedups, for example a 20× or greater speedup.

In some cases, it may be of interest to assume that the error function is not strictly convex, in which case the accuracy may depend on the initial starting point chosen. This may be addressed by accounting for an alternative, but known, pattern of the error function. Alternatively or in combination, two or more initial guess locations may be chosen which tend to span a saddle location where there are two minimum locations on the function. This may double the number of calculations, but still yield a significant improvement in speed.

One technical challenge which may occur using the TRFS system and methods described herein may be removal of distortions and artifacts caused by the slow and oscillatory response of various components in the measurement system. In some instances, algorithms that implement a time-domain deconvolution procedure along with curve-fitting may be used to extract the true fluorescence lifetime measurement despite these distortions and artifacts. However, such algorithms may be computationally intensive and diminish the useful content of the lifetime fluorescence measurement due to simplifying assumptions (such as the order of the polynomial kernel) necessary to implement such algorithms. An alternative algorithm paradigm is described herein which may be much less computationally intensive and may recover nearly the entire lifetime fluorescence measurement. This algorithm may perform deconvolution through simple division and windowing in the Fourier domain. Both the instrument response function (IRF) and raw fluorescence decay measurement may be digitally transformed into the Fourier domains using the Fast Fourier Transform (FFT). Subsequently, division may be performed between the two Fourier domain waveforms in order to obtain the deconvolved result in the Fourier domain. Simply performing this step and transforming back to the temporal domain may be inadequate due to finite bandwidth limitation of the digital sampling system. An additional step of windowing using an apodization window (such as the Blackman window) may be used in order to remove temporal ringing in the deconvolved result. The resultant waveform may then be transformed back into the time domain via the Inverse Fourier Transform (IFFT), thereby yielding the deconvolved result corresponding to the true lifetime fluorescence measurement.

In some instances, deconvolution performed in the Fourier domain as described herein may be followed up by performing a bi-exponential curve fitting to the data in order to avoid over-fitting which may occur due to the sensitivity of the FFT technique to bandwidth. Optional windowing may be performed as described herein before or after curve-fitting to remove temporal ringing in the deconvolved result. The deconvolved results may be transformed back into the time domain via the IFFT as described herein.

### Post-Processing Classification

The calculated fluorescence decay function in the different measured wavelengths may comprise different fluorescence components when characterizing an unknown sample. Each component may have a mono-exponential, bi-exponential, or multi-exponential decay function. In order to classify a complex tissue as tumor or normal, the conventional fluorescence lifetime scalar values may be insufficient. To address this, the decay functions in different wavelength ranges (i.e. for different spectral bands) may be transformed to a two-dimensional spectro-lifetime matrix (SLM) with m×n dimensions, where m is the number of spectral bands used in the measurements and n is the number of decay points used. For example, m may be six when six spectral bands are assessed and n may be three where the different decay points cover fast, average, and slow decay responses. The SLM may be extracted for each responsive fluorescence signal and used as an input to a classification algorithm as described herein.

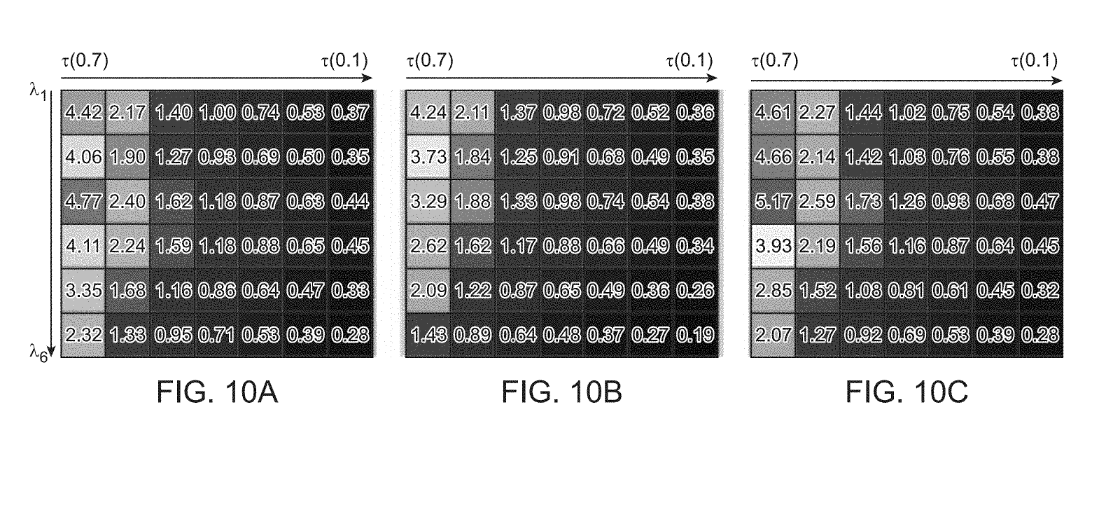

FIG. 10A shows a chart of averaged SLM measured at six different spectral bands (λ₁ to λ₆) and seven decay levels (τ0.1 to τ0.7) for glioma tissue. FIG. 10B shows a chart of averaged SLM measured at six different spectral bands (λ₁ to λ₆) and seven decay levels (τ0.1 to τ0.7) for normal cortex tissue. FIG. 10C shows a chart of averaged SLM measured at six different spectral bands (λ₁ to λ₆) and seven decay levels (τ0.1 to τ0.7) for white matter tissue. The graphs shows the averaged SLM while the variation presents standard deviation. For the training samples, a series of parameters τ(0.1)−τ(0.7) were determined from the detected spectral band decay data for each detection channel (λ₁ to λ₆).

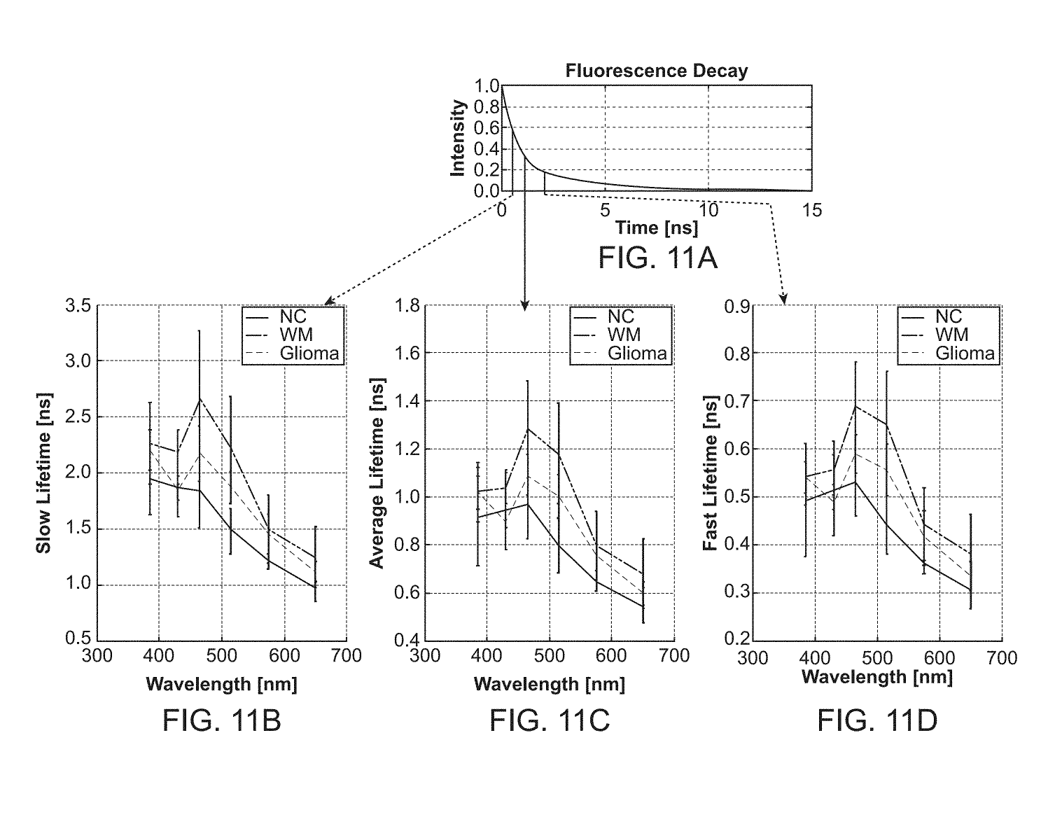

FIG. 11A shows a chart of fluorescence decay profiles of normal cortex, white matter, and glioblastoma (GBM) tissues using six channel TRFS. FIG. 11B shows the spectral signature of the SLM “slow” lifetime for the data shown in FIG. 11A. FIG. 11C shows the spectral signature of the SLM “average” lifetime for the data shown in FIG. 11A. FIG. 11D shows the spectral signature of the SLM “fast” lifetime for the data shown in FIG. 11A. The decay was assessed for each spectral band using Laguerre deconvolution. The parameters τ(0.1)−τ(0.7) were determined for each spectral band of each sample and used to accurately define the fast, normal, and slow components of the fluorescence decay instead of using a full fluorescence decay curve for characterization. Three lifetime values τ(0.2), τ(0.4), and τ(0.6) were extracted from the decay points by crossing the normalized ƒIRF at 0.2, 0.4, and 0.6 intensity levels, respectively, and used as an input to a classification algorithm as representative of slow, normal, and fast decay, respectively. FIGS. 11B to 11D show the lifetime parameters extracted from the training samples at each channel for each decay component. Error bars contain the mean and standard deviation of lifetime values in the six spectral bands. Normal cortex exhibited a faster decay than either white matter or GBM.

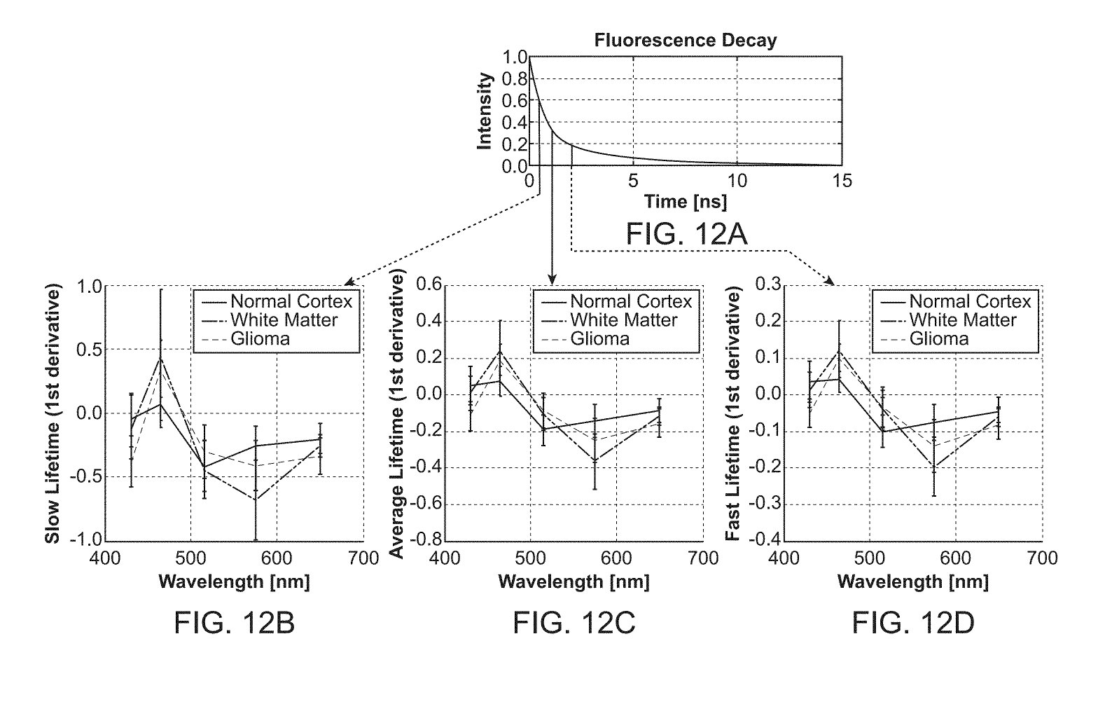

FIG. 12A shows a chart of fluorescence decay profiles of normal cortex, white matter, and glioblastoma (GBM) tissues using six channel TRFS. FIG. 12B shows the first derivative of the SLM spectral signature of the “slow” lifetime for the data shown in FIG. 12A. FIG. 12C shows the SLM spectral signature of the “average” lifetime for the data shown in FIG. 12A. FIG. 12D shows the spectral signature of the “fast” lifetime for the data shown in FIG. 12A. SLM data may contain information about fluorescence lifetimes in different wavelength bands (λ₁ to λ₆). The lifetime values in different bands can offer relative rise or fall between adjacent wavebands. The relative wavelength variations of SLM can be caused by different emission spectra of various fluorescence biomolecules within the unknown sample. Obtaining the derivative of the SLM matrix by λ variations (the dSLM/d λ) may help to magnify the relative wavelength variations of SLM as an input for a classifier as described herein. The decay was assessed for each spectral band using Laguerre deconvolution. The parameters τ(0.1)−τ(0.7) were determined for each spectral band of each sample and used to accurately define the fast, normal, and slow components of the fluorescence decay instead of using a full fluorescence decay curve for characterization. Three lifetime values λ(0.2), λ(0.4), and λ(0.6) were extracted from the decay points by crossing the normalized ƒIRF at 0.2, 0.4, and 0.6 intensity levels, respectively. The first derivative of each of the lifetime values was then calculation for each spectral band. FIGS. 11B to 11D show the first derivative lifetime parameters extracted from the training samples at each channel for each decay component. Error bars contain the mean and standard deviation of first derivative lifetime values in the six spectral bands.

In some instances, classification may be performed by a computer-based algorithm. The computer-based algorithm may for example use machine-learning or neural-networking techniques in order to generate the classifier (i.e., train the classifier) and/or classify the unknown sample. The computer-based algorithm may for example be a machine-learning algorithm that may be trained using various known tissue measurements as a training set. In some instances, classification of the unknown sample may be confirmed by the user, for example using histology, and the now-known sample data may be input into the machine-learning algorithm to further train and fine-tune the classifier.

### Classification Algorithm

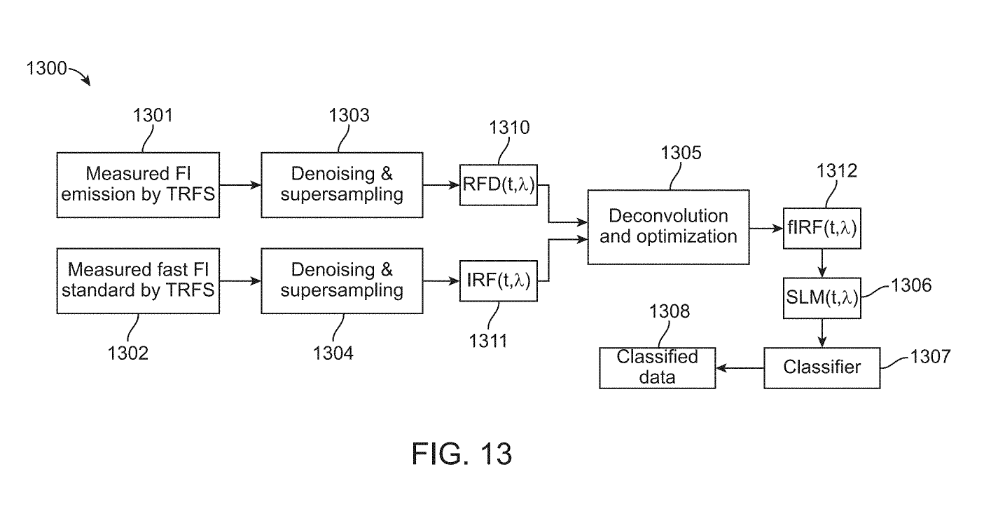

FIG. 13 shows a flowchart of a method 1300 of tissue classification using TRFS SLM data as an input. In order to differentiate two biomolecules (or two tissue types) from their SLM properties, a classifier 1310 may be trained using reference signature SLMs. The reference SLMs can be recorded based on fIRFs confirmed by a gold standard method, for example by histopathological analysis of tissue for identification of normal or tumor tissue. The classifier 1310 may search the SLMs for two or more data groups in order to identify whether there are specific matrix elements with statistically significant difference. A non-limiting example of this test can be performed by a t-test of the null hypothesis (that data in the vectors x and y are independent random samples from normal distributions with equal means and equal but unknown variances). This test may confirm that data with no statistical significance difference between the two groups is not input into the machine learning algorithm. This leaves the SLM elements with maximum discriminating power. Non-limiting examples of classifiers 1310 which may be used to classify an unknown biomolecule based on the confirmed training sets include Principal Component Analysis and/or Linear discriminant Analysis.

At Step 1301, the fluorescence intensity (FI) emission (also referred to herein as the responsive fluorescence signal) of an irradiated sample may be collected by the TRFS system described herein.

At Step 1302, the FI emission of a standard, for example a molecule with known “fast” emission properties or the laser intensity itself, may be collected by the TRFS system as described herein.

At Step 1303, the responsive optical signal from the sample may be pre-processed using the methods for de-noising and/or supersampling to generate raw fluorescence decay data (RFD(t,λ)) 1310 as described herein.

At Step 1304, the responsive optical signal from the standard may be pre-processed using the methods for de-noising and/or supersampling to determine the Instrument Response Function (IRF(t, λ) 1311 as described herein.

At Step 1305, deconvolution and optimization may be performed to remove the IRF(t,λ) 1311 from the raw fluorescence decay data 1310 in order to generate the fluorescence impulse response function (fIRF(t,λ)) 1312.

At Step 1306, the fIRF(t,λ) 1312 may be used to generate a spectro-lifetime matrix (SLM(t,λ)) as described herein.

At Step 1307, the spectro-lifetime matrix may be input into a classifier as described herein.

At Step 1308, the classifier may be used to differentiate the sample between two or more subtypes and output the classified data as described herein.

Although the above steps show method 1300 of tissue classification in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to classify the tissue.

One of more of the steps of the method 1300 may be performed with the system described herein, for example, one or more of the computer or processor. The processor may be programmed to perform one or more of the steps of method 1300, and the program may comprise program instructions stored on a computer readable memory or programmed steps of a logic circuitry such as a programmable array logic of a field programmable gate array, for example.

### Applications

### 1. Measurements of Fluorescence Lifetime In Vivo for Quantification of Fluorophore Concentration in Compound Biomolecules

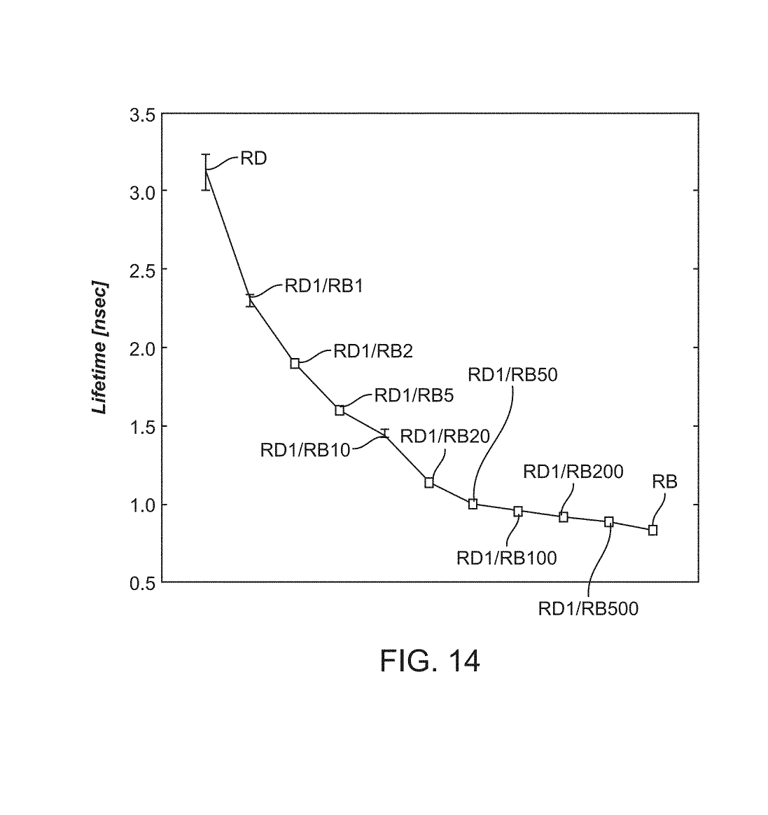

FIG. 14 shows a chart of lifetime variation at different concentrations of Rhodamine B (RD) and Rose Bengal (RB) in 100 μM ethanol solution. The fluorescence decay of a biological sample may for example be used to determine the concentration of known fluorophores. Solutions of varying concentrations of RD and RB were excited with UV light and the responsive fluorescence decay signals were recorded. The concentrations analyzed are shown in Table 2. The fluorescence decay profiles were distinct for each of the various mix concentrations. The different concentrations had unique and distinct lifetime values. These data may thus be used as standards to determine the concentration of an unknown mix of RD and RB for example. Similar dosing or mixing experiments may be used to determine the fluorescence profiles of other fluorophore mixes of interest, for example, to aid in characterization of complex biological samples.

TABLE 2
Ratio of RD and RB in mixes assessed
(shown from left to right in FIG. 14).

| RD | RB |
|---|---|
| 1 | 0 |
| 1 | 1 |
| 1 | 2 |
| 1 | 5 |
| 1 | 10 |
| 1 | 20 |
| 1 | 50 |
| 1 | 100 |
| 1 | 200 |
| 1 | 500 |
| 0 | 1 |

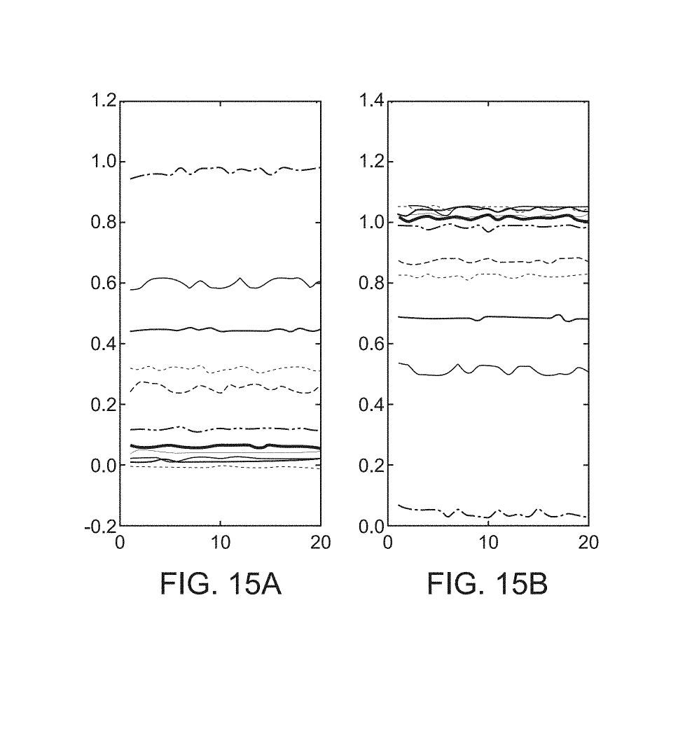

FIGS. 15A and 15B show fitting of the fluorescence impulse response function (fIRF) of the data collected in FIG. 14 to a bi-exponential function (a.exp(−bt)+c.exp(−dt)) where the first exponential coefficients (FIG. 15A) and the second exponential coefficients (FIG. 15B) at multiple measurements correlate with individual concentrations of each component in the mixture. By fitting the resulted fIRF for each concentrations to a bi-exponential function, the relative concentration of RD and RB in each mixture may also be distinguished by their bi-exponential coefficients.

### 2. Noninvasive and Intraoperative Tumor Demarcation

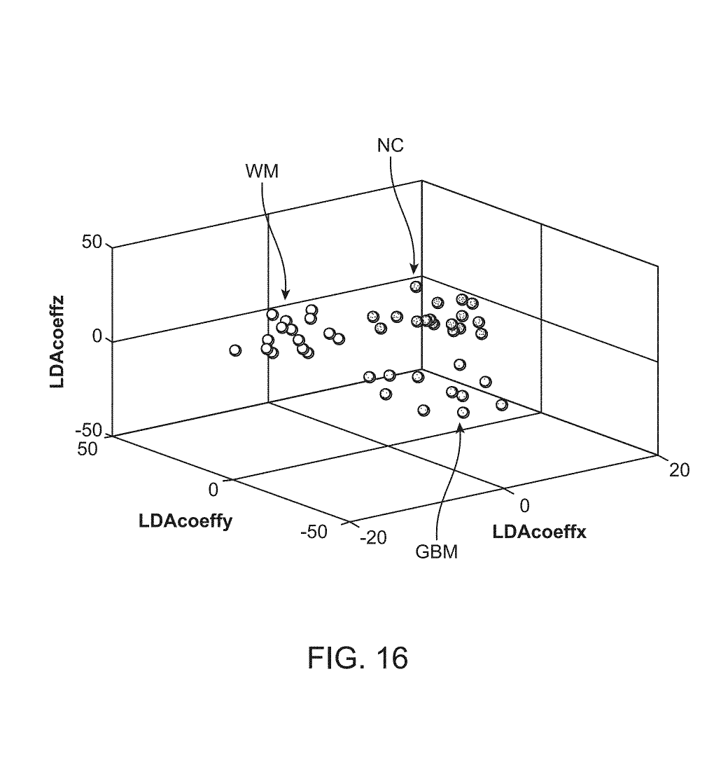

FIG. 16 shows a chart of linear discriminant analysis (LDA) classification for normal cortex, normal white matter, and glioblastoma. LDA can be implemented by a three-group set classifier with the output of the classifier being one of the training groups. Alternatively or in combination, the output may be a result of “true or not true” of the sample belonging to one of the training groups. Three training groups were used to generate FIG. 16: normal cortex (“NC”; n=18), normal white matter (“WM”; n=15), and glioma (“GBM”; n=11). Tissue samples of known tissue type (NC, WM, or GBM) from 5 patients were assayed in vivo to generate the training groups.

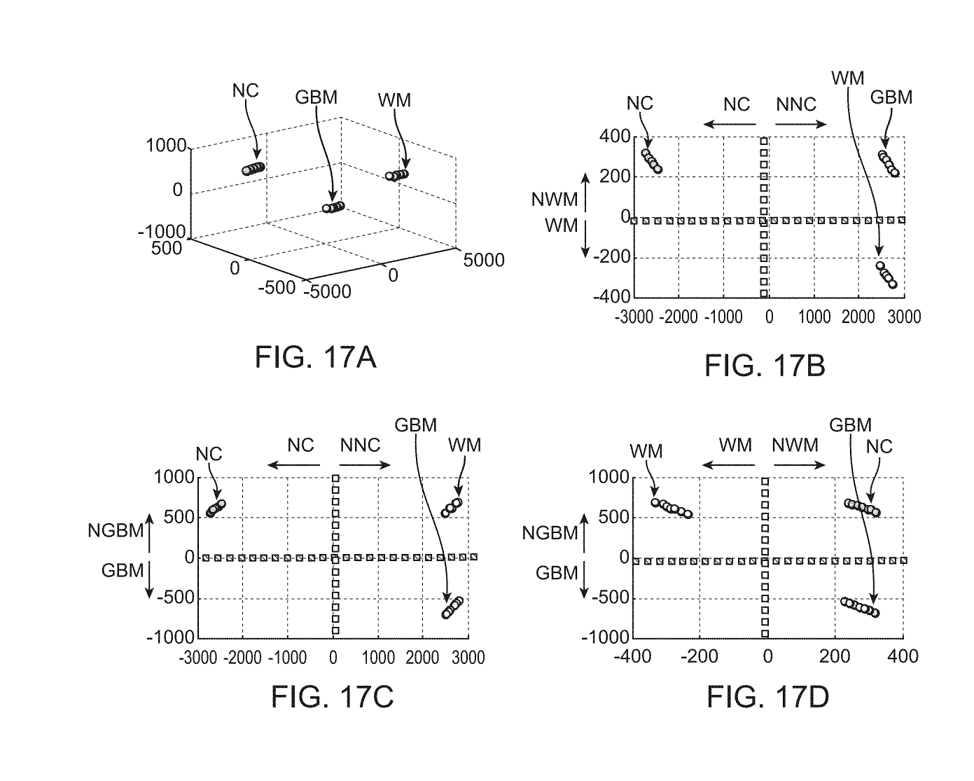

FIG. 17A shows a chart of LDA classification for normal cortex, normal white matter, and glioblastoma. The extracted parameters were used to distinguish between tissue types in the training samples in order to create a classification algorithm. The system generated spectroscopic lifetime (decay) information of the tissue samples which were used as a signature by a machine training algorithm for tissue classification. Linear discriminant analysis (LDA) with a three-group classifier set was used to analyze the fluorescence decay in the six spectral bands collected to maximize the difference in statistical significance between training groups, with the output being sent to either of the training groups. The NC classifier, for example, grouped WM and GBM measurements in the “Not NC” group. The same process was employed for the WM and GBM groups, where “Not WM” comprised NC and GBM and “Not GBM” comprised WM and NC, respectively. These subclassifiers were able to discriminate between training groups and classify the training samples as normal cortex, white matter, or GBM. FIG. 17B shows a chart of “true or not true” LDA classification for white matter versus normal cortex used to generate the chart of FIG. 17A. FIG. 17C shows a chart of “true or not true” LDA classification for normal cortex versus glioblastoma used to generate the chart of FIG. 17A. FIG. 17D shows a chart of “true or not true” LDA classification for white matter versus glioblastoma used to generate the chart of FIG. 17A.

Combination of Monopolar and/or Bipolar Cortical and Subcortical Stimulator Used for Motor Mapping and Language Mapping with TRFS to Enhance Margin Detection and Salvaging Normal Brain Tissue

Electrical stimulation of the brain may be used to provide functional mapping of the brain through direct electrical stimulation of the cerebral cortex and/or subcortical tissue. Cortical and sub-cortical stimulation mapping may be used for a number of clinical and therapeutic applications including pre-operative, intra-operative, and/or post-operative mapping of the motor cortex and language areas in order to prevent unnecessary functional damage during neurosurgery (e.g. for tumor resection). One or more electrodes (which may be within an electrical stimulator probe as described herein) may be placed on the brain in order to test motor, sensory, language, and/or visual function at a target tissue location in the brain. The electrical current from the one or more electrodes may stimulate the target tissue location and produce a responsive electrical response. A physical response (such as a muscle contraction or speech arrest, among others) may also occur when the target tissue is stimulated.

Electrical stimulation may be bi-polar, mono-polar, or both. Bi-polar mapping is more traditionally used for cortical and subcortical mapping as the biphasic stimuli employed may mitigate potential adverse effects of electrical stimulation which may occur with mono-polar stimulation. That said, the advent of better constant-current generators has led to safer mono-polar, monophasic stimulators which may also be of interest.

The TRFS methods and systems described herein may provide a surgeon with a (near) real-time, intraoperative tool (which may also be used pre- and/or post-operative) for interrogation and identification of brain tumor margins, for example. This may be achieved by differentiating between distinct fluorescence decay signatures characteristic of normal brain tissue and tumor tissue as described herein. Alternatively or in combination with TRFS methods and systems described herein, the brain tissue may be functionally interrogated, for example by electrical stimulation and mapping of the brain, in order to enhance the diagnostically-relevant information obtained using TRFS. Mapping of the normal brain, for example for motor and/or speech functions, may inform surgical resections of tumors by alerting the surgeon to functionally important areas of the brain which may need to be avoided during surgery.

The TRFS systems and methods described herein may be combined with electrical mapping of the brain in order to more accurately identify and preserve the functioning brain in (near) real-time. TRFS may be used to interrogate the biochemical natures of the brain tissue while an electrical stimulation may be used to interrogate the electrical and functional aspects of the brain tissue. Alternatively or in combination, TRFS may be used to interrogate exogenous fluorescently-labeled molecules (such as fluorescently-labeled drugs) at unconventional depths within a target tissue. When combined, TRFS and electrical stimulation may provide more information intraoperatively than traditional imaging methods, such as MRI and ultrasound, which may only provide structural information. Such information may lead to more complete and safer resection of brain tumors while important parts of the normal brain are identified and avoided or protected.

The TRFS methods and systems described herein may optionally be combined with electrical stimulation in order to enhance tissue detection and classification. The system may optionally comprise an electrical stimulator. When the biological sample comprises brain tissue, an electrical stimulator may comprise one or more of a mono-polar or bi-polar cortical and subcortical stimulator. The biological sample may comprise cortical and/or subcortical tissue. The electrical stimulator may electrically stimulate the biological sample to produce a responsive optical signal in response. The electrical stimulator may be configured for recording a responsive electrical signal indicative of electrical function activity of the biological sample. In some embodiments, a module configured for recording the electrical function activity of the biological sample may be used for obtaining the responsive electrical signal. For example, the electrical stimulator may comprise a cortical stimulator from, or adapted from, the OCS2 Ojemann Cortical Stimulator available from Integra LifeSciences. The electrical stimulator may comprise a probe. The probe may be configured to be handheld. The probe may comprise a handheld probe. The probe may be robotically-controlled, for example with a commercially-available robotic surgery system. The probe to provide the electrical stimulation may function to provide the TRFS interrogation and/or tissue ablation as described above.

Any of the systems, devices, or probes described herein may further comprise an ablation element to ablate a target tissue of the biological sample. The target tissue may be ablated or removed in response to characterization of the target tissue as described herein. The ablation element may be configured to apply one or more of radiofrequency (RF) energy, thermal energy, cryo energy, ultrasound energy, X-ray energy, laser energy, or optical energy to ablate a target tissue. The ablation element may be configured to apply laser or optical energy to ablate the target tissue. The ablation element may comprise the excitation signal transmission element of the TRFS system described herein. The ablation element may comprise any of the probes described herein. The probe may be configured to ablate the target tissue, irradiate the biological sample with the light pulse, stimulate the brain, and/or collect the responsive fluorescence signal (in any order desired). The combination of ablation, time-resolved fluorescence spectroscopy, and/or electrical stimulation may be used to determine which tissue should be ablated prior to ablation, to monitor ablation as it occurs, and/or to confirm that the correct tissue was ablated after ablation ends. In some instances, commercially-available ablation probes may be modified to collect a fluorescence signal from the tissue as described herein and used to generate time-resolved fluorescence spectroscopy data as described herein.

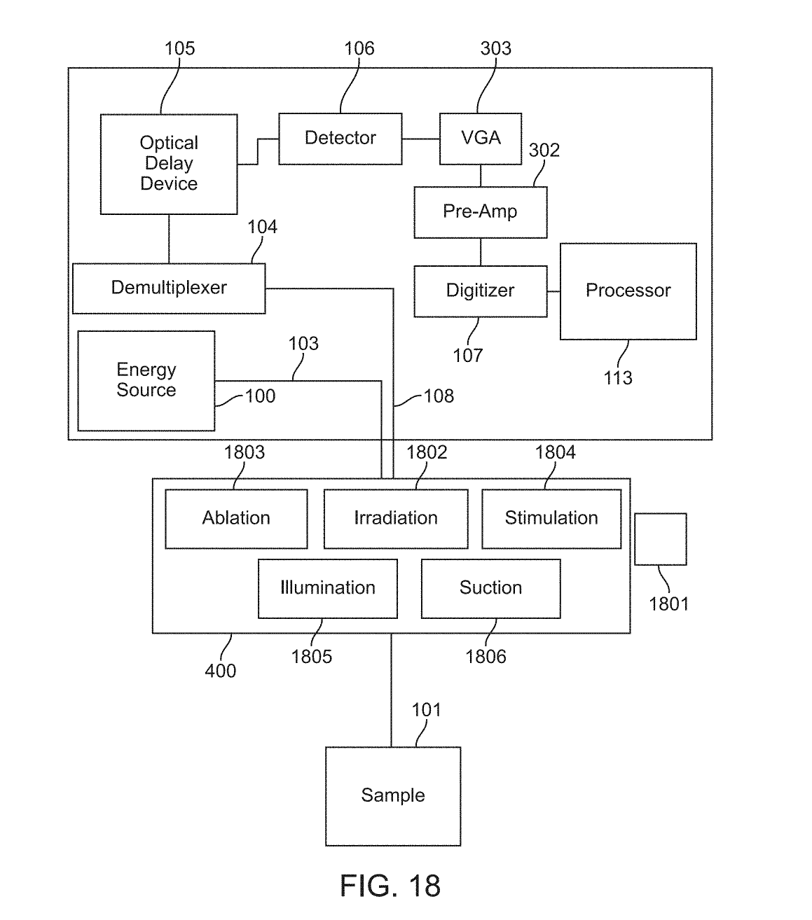

FIG. 18 shows a schematic of a TRFS system. The system may be used to characterize a biological sample 1800 using real-time, or near real-time, time-resolved fluorescence spectroscopy. The system may be substantially similar to other systems described herein and the elements of the system may be substantially similar to such elements described herein. The system may comprise an excitation signal transmission element 103, a light source 100, at least one signal collection element 108, an optical assembly such as a demultiplexer 104, and an optical delay device or element 105. The system may further comprise one or more of a detector 106, a digitizer 107, a computer or processor 113, a voltage-gated attenuator 302, or a pre-amplifier 302. The system may comprise other elements which are not shown but have been described herein, such as a one or more of a photodiode, a detector gate, or a trigger synchronization mechanism 102. In some instances, at least a portion of the excitation signal transmission element 103 and the at least one signal collection element 108 may comprise a handheld or robotically-controlled probe 400 which may operably coupled to the rest of the system components. The probe 400 may comprise a handheld probe. The probe 400 may be configured to be handheld by the hand 1801 of an operator, for example a surgeon. The probe 400 may be robotically-controlled (not shown), for example with a commercially-available robotic surgery system.

The probe 400 may be configured to irradiate 1802 the biological sample 101 and collect the responsive fluorescence signal for TRFS. The sample 101 may be irradiated with a light pulse from the energy source 100 carried to the sample 101 by the excitation signal transmission element 103 as described herein. The probe 400 may collect the responsive fluorescence signal using the at least one signal collection element 108 and direct the signal toward the demultiplexer 104 as described herein. The demultiplexer 104 may split the responsive fluorescence signal into one or more spectral bands and the optical delay device may apply one or more time delays to the one or more spectral bands as described herein. The time-delayed spectral bands may then be detected by the detector 106, digitized by the digitizer 107, and recorded by the computer 113 as described herein. Alternatively or in combination, the probe 400 may be configured to ablate 1803 the tissue as described herein. For example, the probe 400 may be configured to irradiate the biological sample 101 with the light pulse and collect the responsive fluorescence signal which may then be used to characterize the sample 1800. In response to characterization of the tissue as abnormal, for example as tumor tissue, the probe 400 may then be used to ablate 1803 the area of the sample 101 identified as abnormal. Alternatively or in combination, the probe 400 may be configured to provide electrical stimulation 1804 to the tissue as described herein. For example, the probe 400 may be configured to irradiate 1802 the sample 101 and electrically stimulation 1804 the sample.

The probe 400 may be configured to ablate 1803 the target tissue, irradiate 1802 the biological sample 101 with the light pulse, stimulate 1804 the brain 101, and/or collect the responsive fluorescence signal (in any order desired) as described herein. The combination of ablation 1803, time-resolved fluorescence spectroscopy 1802, and/or electrical stimulation 1804 may be used to determine which tissue should be ablated prior to ablation, to monitor ablation as it occurs, and/or to confirm that the correct tissue was ablated after ablation ends. In some instances, commercially-available ablation probes may be modified to collect a fluorescence signal from the tissue as described herein and used to generate time-resolved fluorescence spectroscopy data as described herein. In some instances, the probe 400 may be integrated with an illumination source 1805 in order to provide the user/surgeon with illumination of the sample 101. In some instances, the probe 400 may be integrated with a suction cannula 1806, for example to allow for (near) real-time spectroscopy-guided surgical resection.

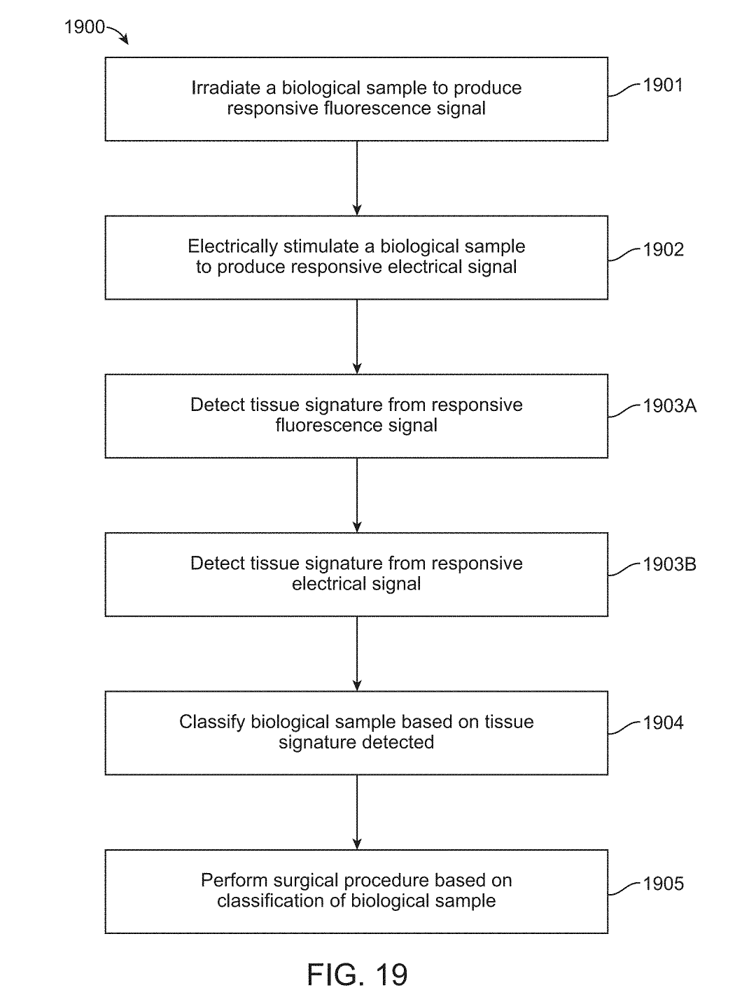

FIG. 19 shows a flowchart of an exemplary method 1800 of tissue classification.

At Step 1901, a biological sample may be irradiated to produce a responsive fluorescence signal as further described above and herein. The responsive fluorescence signal may comprise time-delayed spectral bands. The biological sample may be imaged using TRFS to produce the responsive fluorescence signal.

At Step 1902, the biological sample may be electrically stimulated to produce a responsive electrical signal as further described above and herein. The responsive electrical signal may comprise electrical function data, such as electrical activity of the biological sample in response to the electrical stimulus.

At Step 1903A, the tissue signature may optionally be detected using the responsive fluorescence signal as further described above and herein. The tissue signature may for example be a normal tissue signature. The tissue signature may for example be an abnormal tissue signature, for example a tumor tissue signature.

At Step 1903B, the tissue signature may alternatively or in combination be detected using the responsive electrical signal comprising electrical function data, such as in any of the ways further described above and herein. The tissue signature may for example be a normal tissue signature. The tissue signature may for example be an abnormal tissue signature, for example a tumor tissue signature. For example, the tissue signature may be normal cortex, white matter, or glioma, as described in FIGS. 16-17D.

At Step 1904, the biological sample may be classified based on the detected tissue signature, such as in any of the ways further described above and herein. The biological sample may for example be classified as normal tissue based detection of a normal tissue signature. The biological sample may for example be classified as tumor tissue based detection of a tumor tissue signature. In some instances, classification may be performed by a computer-based algorithm. The computer-based algorithm may for example use machine-learning or neural-networking techniques in order to generate the classifier (i.e. train the classifier) and/or classify the unknown sample as described herein.

At Step 1905, the classification information may be used to inform a surgical procedure, such as in any of the ways further described above and herein. For example, if a tissue is identified as normal tissue, the tissue may be preserved during the surgical procedure. If a tissue is identified as tumor tissue, the tissue may be removed during the surgical procedure, for example by surgical ablation as further described herein.

Although the above steps show method 1900 of tissue classification in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to classify the tissue.

One of more of the steps of the method 1900 may be performed with the system described herein, for example, one or more of the computer or processor. The processor may be programmed to perform one or more of the steps of method 1900, and the program may comprise program instructions stored on a computer readable memory or programmed steps of a logic circuitry such as a programmable array logic of a field programmable gate array, for example.

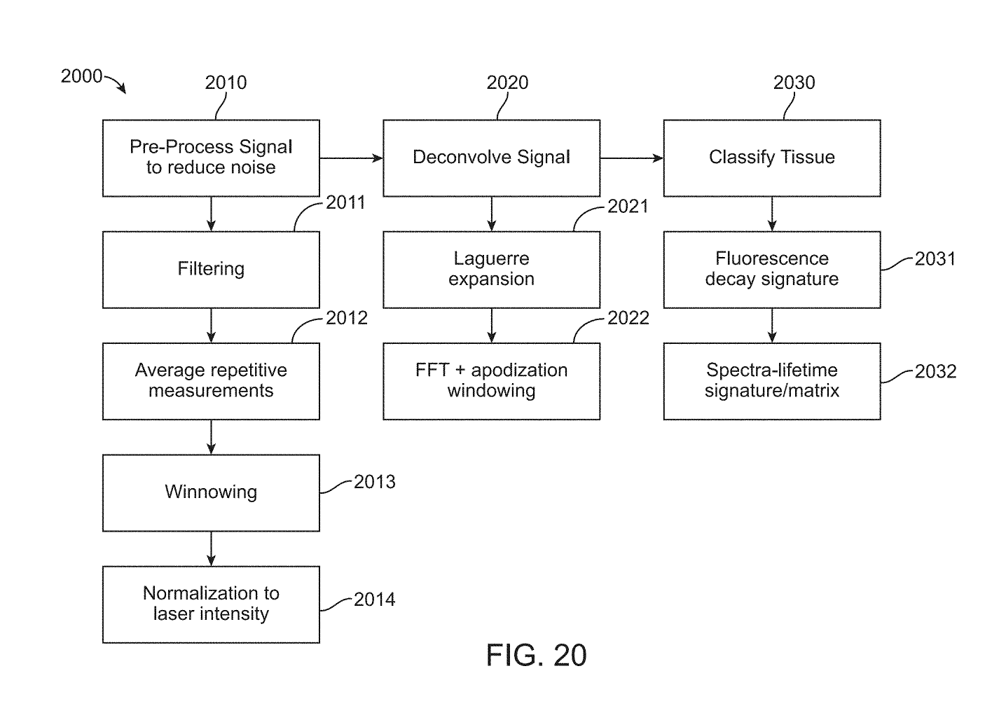

FIG. 20 shows a flowchart of an exemplary method 2000 of tissue classification.

The method 2000 may comprise three main stages: i) signal preprocessing 2010, ii) deconvolution optimization 2020, and iii) post processing classification 2030 to identify a tissue type of a biological sample. These steps may comprise one or more sub-steps as described herein.

At Step 2010, a responsive fluorescence signal may be pre-processed in order de-noise the raw fluorescence decay data as described herein. Pre-processing may comprise one or more substeps. For example, pre-processing may comprise filtering (Step 2011), averaging (Step 2012), winnowing (Step 2013), normalization (Step 2014), or any combination thereof.

At Step 2011, the responsive fluorescence signal may be pre-processed to reduce noise by filtering the signal as described herein. The signal may for example be filtered using a Savitzky-Golay filter to remove high frequency noise as described herein.

At Step 2012, the responsive fluorescence signal may be averaged over multiple repetitive measurements in order to reduce signal variation and discrepancies in the signal as described herein. As the number of repetitions increases, the lifetime standard variation may be reduced as described herein.

At Step 2013, the responsive fluorescence signal may be winnowed to reduce noise as described herein. One of more outliers in the data may be removed from a group of measurements in the raw fluorescence decay data which share the same temporal point. This may then be repeated for each temporal point as described herein.

At Step 2014, the responsive fluorescence signal may be normalized to the laser intensity used to generate the signal in order to improve the accuracy of the responsive fluorescence signal as described herein. The intensity of each excitation light pulse may be recorded and may be used to normalize the responsive fluorescence signal of each pulse as described herein. The intensity of the light pulse may for example be recorded by a photodiode as described in FIG. 4B. Alternatively or in combination, the intensity of the light pulse may be determined from a spectral band generated by the demultiplexer which contains wavelengths at or about the excitation wavelength (for example spectral band 111a) as described in FIG. 2.

At Step 2020, the pre-processed raw fluorescence decay data may be deconvolved and optimized as described herein. Deconvolution may comprise one or more substeps. For example, deconvolution optimization may comprise performing a Laguerre expansion of kernels on the pre-processed data (Step 2021), performing a Fast Fourier Transform (FFT) on the pre-processed data with apodization windowing and/or curve-fitting (Step 2022), or any combination thereof.

At Step 2021, the pre-processed raw fluorescence decay data may be deconvolved by applying a Laguerre expansion as described herein. Optionally, de-convolving the pre-processed raw fluorescence data may comprise optimizing one or more of a Laguerre parameter or a temporal shift of the Laguerre expansion. Optimizing the one or more of the Laguerre parameter or the temporal shift may comprise implementing an iterative search method. For example, a global search method and/or a random walk method may be used to obtain optimized α and temporal shift values as described herein.

At Step 2022, the pre-processed raw fluorescence decay data may be deconvolved by in the Fourier domain after transformation using the FFT as described herein. An apodization window (such as the Blackman window) may be used in order to remove temporal ringing in the deconvolved result as described herein. Alternatively or in combination, the deconvolved result may be fit to a bi-exponential curve as described herein to avoid the over-fitting which may occur due to the sensitivity of the FFT technique to bandwidth. The data may then be transformed back into the time domain via the Inverse Fourier Transform (IFFT) as described herein.

At Step 2030, the tissue may be classified in response to the deconvolved tissue signal. Tissue classification may comprise one or more substeps. For example, tissue classification may comprise classifying the tissue in response to a true fluorescence decay signature generated by pre-processing and deconvolution (Step 2031), classifying the tissue in response to a spectro-lifetime signature or matrix generated from the true fluorescence decay data (Step 2032), or any combination thereof. In some instances, classification may be performed by a computer-based algorithm. The computer-based algorithm may for example use machine-learning or neural-networking techniques in order to generate the classifier (i.e., train the classifier) and/or classify the unknown sample as described herein.

At Step 2031, the true fluorescence decay signature may be used to classify the tissue as described herein. Classifiers which may be used to classify an unknown biomolecule or tissue based on confirmed training sets include Prinicpal Component Analysis and/or Linear Discriminant analysis as described herein. For example, the true fluorescence decay signature generated using the methods described herein may be input into a classifier for classification as described herein.

At Step 2032, the true fluorescence decay data may be used to generate a spectro-lifetime signature or matrix as described herein. The spectro-lifetime signature or matrix may be used to classify the tissue as described herein. Classifiers which may be used to classify an unknown biomolecule or tissue based on confirmed training sets include Principal Component Analysis and/or Linear Discriminant analysis as described herein. For example, the spectro-lifetime matrix generated using the methods described herein may be input into a classifier for classification as described herein.

Although the above steps show method 2000 of tissue classification in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to classify the tissue.

One of more of the steps of the method 2000 may be performed with the system described herein, for example, one or more of the computer or processor. The processor may be programmed to perform one or more of the steps of method 2000, and the program may comprise program instructions stored on a computer readable memory or programmed steps of a logic circuitry such as a programmable array logic of a field programmable gate array, for example.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

Many variations and alternative elements have been disclosed in embodiments of the present invention. Still further variations and alternate elements will be apparent to one of skill in the art. Among these variations, without limitation, are the selection of constituent modules for the inventive methods, compositions, kits, and systems, and the various conditions, diseases, and disorders that may be diagnosed, prognosed, or treated therewith. Various embodiments of the invention can specifically include or exclude any of these variations or elements.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for classifying or characterizing a biological sample, the method comprising:
characterizing the biological sample in response to a responsive fluorescence signal and a responsive electrical signal,
wherein the responsive fluorescence signal is produced by the biological sample in response to the biological sample being irradiated with a light pulse, and
wherein the responsive electrical signal is produced by the biological sample in response to electrical stimulation.

2. The method as in claim 1, wherein the biological sample comprises cortical or subcortical tissue.

3. The method as in claim 1, wherein the light pulse comprises an excitation signal at a predetermined wavelength.

4. The method as in claim 1, wherein the responsive fluorescense signal comprises one or more of a spectral signature, spectro-lifetime signature, spectro-lifetime matrix, or fluorescence decay signature, and wherein the biological sample is characterized in response to the one or more of the spectral signature, spectro-lifetime signature, spectro-lifetime matrix, or fluorescence decay signature.

5. The method as in claim 1, wherein characterizing the biological sample in response to the responsive fluorescence signal and the responsive electrical signal comprises splitting the responsive fluorescence signal into a plurality of spectral bands and characterizing the biological sample in response to the spectral bands.

6. The method as in claim 1, wherein characterizing the biological sample in response to the responsive fluorescence signal and the responsive electrical signal comprises determining a concentration of a biomolecule in response to the responsive fluorescence signal.

7. The method as in claim 1, wherein the biological sample is characterized as normal, benign, malignant, scar tissue, necrotic, hypoxic, viable, non-viable, or inflamed.

8. The method as in claim 1, wherein the biological sample comprises brain tissue.

9. The method as in claim 1, wherein the biological sample comprises a target tissue, and wherein the target tissue is ablated.

10. The method as in claim 9, wherein the target tissue is removed or ablated in response to the characterizing of the biological sample.

11. The method as in claim 9, wherein the target tissue is ablated by applying one or more of radiofrequency (RF) energy, thermal energy, cryo energy, ultrasound energy, X-ray energy, laser energy, or optical energy to the target tissue.

12. The method as in claim 9, wherein the target tissue is ablated with a probe, the probe being configured to radiate the biological sample with the light pulse and collect the responsive fluorescence signal.

13. The method as in claim 1, wherein the biological sample is radiated with the light pulse and electrically stimulated with a probe.

14. The method as in claim 1, wherein the biological sample is electrically stimulated with one or more of a bi-polar or mono-polar cortical and subcortical stimulator.

15. A method for classifying or characterizing a biological sample, the method comprising:
pre-processing raw fluorescence decay data, wherein the raw fluorescence decay data is generated from a responsive fluorescence signal collected from a biological sample exposed to a light excitation signal at a predetermined wavelength; and
de-convolving the pre-processed raw fluorescence decay data to remove an instrument response function therefrom, thereby generating true fluorescence decay data,
wherein the biological sample is characterized in response to the true fluorescence decay data.

16. The method as in claim 15, wherein pre-processing the raw fluorescence decay data comprises removing high frequency noise.

17. The method as in claim 15, wherein pre-processing the raw fluorescence decay data comprises averaging multiple repetitive measurements in the raw fluorescence decay data.

18. The method as in claim 15, wherein pre-processing the raw fluorescence decay data comprises removing one or more outliers from a group of measurements in the raw fluorescence decay data, the group of measurements sharing a same temporal point.

19. The method as in claim 18, further comprising repeating the removing of one or more outliers for a plurality of measurement groups at different temporal points.

20. The method as in claim 15, wherein de-convolving the pre-processed raw fluorescence data comprises applying a Laguerre expansion to the pre-processed raw fluorescence data.

21. The method as in claim 20, wherein de-convolving the pre-processed raw fluorescence data comprises optimizing one or more of a Laguerre parameter or a temporal shift of the Laguerre expansion.

22. The method as in claim 21, wherein optimizing the one or more of the Laguerre parameter or the temporal shift comprises implementing an iterative search method.

23. The method as in claim 15, wherein de-convolving the pre-processed raw fluorescence data comprises dividing and windowing one or more of the raw fluorescence decay data or the instrument response function in the Fourier domain.

24. The method as in claim 15, wherein the biological sample is characterized by generating a fluorescence decay function from the true fluorescence decay data and transforming the fluorescence decay function into a Spectro-Lifetime matrix.

25. The method as in claim 24, wherein the biological sample is characterized by comparing the Spectro-Lifetime matrix for the biological sample to a reference Spectro-Lifetime matrix for a tissue characterization.

26. The method as in claim 15, the biological sample is characterized as normal, benign, malignant, scar tissue, necrotic, hypoxic, viable, non-viable, or inflamed.

27. The method as in claim 15, wherein characterizing the biological sample comprises determining a concentration of a biomolecule in the biological sample.

28. The method as in claim 15, wherein the biological sample is treated in response to the characterizing of the biological sample.

29. The method as in claim 15, wherein the biological sample comprises brain tissue.

30. A method for classifying or characterizing a biological sample, the method comprising:
recording an intensity of an excitation light pulse, wherein a biological sample is irradiated with the excitation light pulse at a predetermined wavelength to cause the biological sample to produce a responsive fluorescence signal; and
normalizing a responsive fluorescence signal in response to the recorded intensity of the excitation light pulse,
wherein the biological sample is characterized in response to the normalized responsive fluorescence signal.
